(19) United States
(12) Patent Application Publication
(10) Pub. No.: US 2006/0030616 A1
(43) Pub. Date: Feb. 9, 2006

(54) Anhydride modified cantharidin analogues useful in the treatment of cancer

(76) Inventors: Adam McCluskey, Mayfield (AU); Jennette A. Sakoff, Belmont (AU); Stephen Ackland, New Lambton Heights (AU); Alistair T.R. Sim, Callaghan (AU)

Correspondence Address: PILLSBURY WINTHROP SHAW PITTMAN LLP, ATTENTION: DOCKETING DEPARTMENT, P.O BOX 10500, McLean, VA, 22102, US

(21) Appl. No.: 11/240,971

(22) Filed: Sep. 30, 2005

Related U.S. Application Data
(63) Continuation of application No. 10/703,336, filed on Nov. 7, 2003, now abandoned.
Application No. 10/703,336 is a continuation of application No. 09/743,841, filed on May 9, 2001, now abandoned.

(30) Foreign Application Priority Data
Jul. 14, 1998 (AU) PP 4665

(51) Int. Cl. C07D 333/74; C07D 307/94; A61K 31/407; A61K 31/365; A61K 31/381

(52) U.S. Cl. 514/443; 514/468; 514/411; 548/431; 549/44; 549/237

(57) ABSTRACT

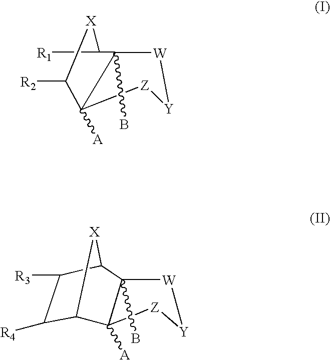

Anhydride modified cantharidin analogues useful in the treatment of certain forms of cancer also methods for the screening for anti-cancer activity of these analogues and/or their ability to sensitise cancer cells to cancer treatment. The modified cantharidin analogues have structure (I) or (II), wherein R₁, R₂, R₃ and R₄ are H, aryl or alkyl; X is O, N or S; Y is O, S, NH, NR; R is alkyl or aryl; A and B are H or CH₃; W and Z are CHOH or C═O. These compounds inhibit protein phosphatase.

### TECHNICAL FIELD

This invention relates to compounds useful in the treatment of certain forms of cancer; processes for producing these compounds; methods of treatment using these compounds per se, methods of treatment using these compounds which methods also increase the sensitivity of cancer cells to other treatments; methods of screening these compounds for anti-cancer activity; and methods of screening these compounds for anti-cancer activity and/or ability to sensitise cancer cells to other methods of treatment More particularly, the compounds are specific inhibitors of protein phosphatases 1 and 2A.

### BACKGROUND ART

Protein Phosphatase Inhibitors and the Abrogation of Cell Cycle Checkpoints

The regulation of protein phosphatases is integral to the control of many cell processes, including cell growth, transformation, tumour suppression, gene transcription, apoptosis, cellular signal transduction, as neurotransmission, muscle contraction, glycogen synthesis, and T-cell activation. The role of protein phosphatases in many of these processes is often mediated via alterations in the cell cycle. Cell cycle progression is tightly regulated to ensure the integrity of the genome. During cell division it is imperative that each stage of the cell cycle be completed before entry into the next, and this is achieved through a series of checkpoints. The cell cycle can be broken down into four phases, the first gap (G₁), is followed by a phase of DNA synthesis (S-phase); this is followed by a second gap (G₂) which in turn is followed by mitosis (M) which produces two daughter cells in G₁. There are two major control points in the cell cycle, one late in G₁, and the other at the G₂/M boundary. Passage through these control points is controlled by a universal protein kinase, cdk1. The kinase activity of cdk1 is dependant on phosphorylation and the association with a regulatory subunit, cyclin B. The periodic association of different cyclins with different cyclin dependent kinases (cdk) has been shown to drive different phases of the cell cycle; thus cdk4-cyclin D1 drives cells through mid G₁, cdk2-cyclin E drives cells in late G₁, cdk2-cyclin A controls entry into S-phase and cdc2-cyclin B drives the G₂/M transition (O'Connor, 1996, 1997).

Following DNA damage induced by chemotherapy or radiation treatment these checkpoints are responsible for halting cell cycle progression in G₁. S and/or G₂ phases (O'Connor, 1996). The cell undergoes a cell cycle arrest so that the damaged DNA can be repaired before entry into S phase or mitosis. The phase at which the cell cycle is halted will depend upon the type of DNA damaging agent used and the point during the cell cycle that the damage was incurred (O'Connor, 1997). The cell cycle is controlled and regulated by an intricate phosphorylation network (Stein et al., 1998). More particularly, activation of cdk/cyclin complexes requires the phosphorylation of a conserved threonine residue, which are catalysed by CAK kinase, as well as the removal of inhibitory phosphorylations by the phosphatase cdc25. Cdc25 is only active in its phosphorylated form. Therefore, protein phosphatase 2A (PP2A) can inhibit the activation of cdk/cyclin complexes by inhibiting CAK activity and by dephosphorylating cdc25. The G₁/S checkpoint is predominantly regulated by the cdk/cyclin D/E complex that mediates its effects by phosphorylating and inactivating the tumour suppressor protein retinoblastoma (pRb). The phosphorylation of pRb prevents it from interacting with the S-phase transcription factor E2F. E2F controls the transcription of proteins needed for DNA synthesis and entry into S-phase including thymidylate synthase. Accordingly, the inactivation of pRb by phosphorylation permits entry into the S-phase and vice versa. However, protein phosphatase 1 (PP1) can dephosphorylate pRb and inhibit the cell cycle (Durfee et al., 1993). Thus, PP1 and PP2A are both negative regulators of the cell cycle. Inhibition of PP1 and PP2A would abrogate these checkpoints and prematurely force cells through the cell cycle.

Serine/threonine phosphatases, which are responsible for protein dephosphorylation, comprise a unique class of enzymes consisting of four primary subclasses based on their differences in substrate specificity and environmental requirements. Of the serine/threonine phosphatases, protein phosphatases 1 and 2A (PP1 and PP2A, respectively) share sequence identity between both enzyme subunits (50% for residues 23-292; 43% overall), are present in all eukaryotic cells and are together responsible for 90% of all cellular dephosphorylation. Knowledge of structure and subsequent correlation of binding function for both PP1 and PP2A would therefore provide a vital link toward understanding the biochemical role of these enzymes. A goal of the medicinal chemist is the development of potent and selective inhibitors of these protein phosphatases.

The natural toxins, okadaic acid, calyculin A, microcystin-LR and tautomycin are representative of a structurally diverse group of compounds that are all potent protein phosphatase 1 (PP1) and 2A (PP2A) inhibitors. Okadaic acid is more specific for PP2A (IC₅₀ 1 nM) than PP1 (IC₅₀ 60 nM), while calyculin is slightly more specific for PP1 (IC₅₀ 0.5-1.0 nM) than PP2A (IC₅₀ 2 nM). All of these phosphatase inhibitors are known to abrogate cell cycle checkpoints, particularly the G₂ checkpoint of the cell cycle and induce cellular mitoses (Yamashita et al., 1990). Abrogation of the G₂ checkpoint means that the cell does not have the capacity to detect DNA damage or malformation of the genome prior to entry into mitosis. Therefore, cells which have a deficient G₂ checkpoint are unstable; and incapable of detecting DNA damage, initiating G₂ arrest, or undergoing DNA repair. Such cells enter the mitotic stage of the cell cycle prematurely with malformed spindles. The abrogation is of the G₂ checkpoint in the cell cycle by okadaic acid is mediated via the activation of cdc2/H1 kinase, the major mitotic inducer, and results in a premature mitotic state (Yamashita et al. 1990). Although okadaic acid is known as a tumour promoter, in some cell types, it has been shown to revert the phenotype of oncogene-transformed cells to that of normal cells, and to inhibit neoplastic transformation of fibroblasts (Schonthal, 1991).

Furthermore, okadaic acid has been shown to selectively enhance the cytotoxicity of vinblastine and the formation of apoptotic cells, in HL60 cells which are p53 nul (Kawamura, 1996). Interestingly, calyculin enhances irradiation killing in fibroblast cells at doses that are non toxic when given as a single treatment. (Nakamura and Antoku, 1994). Data also shows that okadaic acid can abrogate the G₁/S checkpoint of the cell cycle. In this context, okadaic acid has been shown to overide the S-phase checkpoint and accelerate progression of G₂-phase to induce premature mitosis (Gosh et al., 1996). In addition, okadaic acid has been shown to significantly increase the fraction of quiescent cells entering the S-phase via modifications in the phosphorylation state of pRb (Lazzereschi et al. 1997). Other studies have shown that the hyperphosphoryation state of pRb forces cells prematurely into S-phase and pRb can be kept in a phosphorylated state via protein phosphate inhibition (Herwig and Strauss, 1997). Cells lacking functional pRb show increased apoptosis and cytotoxicity following 5-fluorouracil and methotrexate treatment (Herwig and Strauss, 1997). We propose that cell death would be substantially enhanced in cells forced to enter the S-phase prematurely (via G₁ checkpoint abrogation) and which were lacking key S-phase components such as dTMP (via TS inhibition).

The okadaic acids class of compounds, with the exceptions of okadaic acid, cantharidin (Honaken) and thyrisferyl 23-acetate (Matszawa et. al) (being PP2A selective) exhibit poor selectivity. Furthermore, the concentration of PP1 and PP2A inside cells is such that high concentrations of these inhibitors are required to generate a response in vivo resulting in the loss of effectiveness of any in vitro selectivity (Wang).

Cantharidin (exo.exo-2.3-dimethyl-7-oxobicyclo[2.2.1]heptane-2,3-dicarboxylic acid anhydride), is a major component of the Chinese blister beetles: Mylabris phaleraia or M. cichorii)(Yang; Cavill et. al). The dried body of these beetles has been used by the Chinese as a natural remedy for the past 2000 years. Although Western medicine decreed cantharidin to be too toxic in the early 1900's (Goldfarb et. al) its purported aphrodisiac qualities (the active ingredient of “Spanish Fly”), and its widespread occurrence in cattle feed still results in numerous human and livestock poisonings (Schmitz).

Li and Casida, and previous work in this laboratory (McCluskey et. al) (and more recently Pombo-Villar, Sodeoka) has assisted in the delineation of certain features crucial for inhibition of PP2A by cantharidin analogues (FIG. 1). However the corresponding picture for PP1 is not so clear, the majority of data refers to possible interactions with the known crystal structures, and in some cases the inhibition values for PP1 are not reported.

Involvement of Tumour Suppressor Gene p53

The most commonly mutated gene in human cancers is the tumour suppressor gene p53, which is abnormally expressed in more than 50% of tumours. The development of chemotherapeutic agents which selectively target cancer cells with mutant p53 is certainly desirable, for two main reasons. Firstly, cells that have an abnormal p53 status are inherently resistant to conventional chemotherapy and produce the more common, and more aggressive tumours such as colon carcinoma and non small cell lung cancer. Secondly, a chemotherapy regime that targeted only those cells with a mutant p53 phenotype would potentially produce fewer side effects since only the cancer cells would be killed and not the p53 proficient normal healthy cells.

### DISCLOSURE OF THE INVENTION

In relation to the discussion above, the present inventors believed that the replacement of the ether O atom of the anhydride with N or S (as N—H and N—R, where R=alkyl or aryl) would allow them to probe the H-bonding requirements of this region of cantharidin analogues. Previous studies in their laboratory had shown limited tolerance for modification of the 7-oxa position. An ability to modify these heteroatoms is crucial to the development of selective inhibitors based on this simple skeleton.

There is not, at present, an inhibitor with either absolute specificity or high enough selectivity which renders the inhibitor effectively specific in vivo.

It has surprisingly been found that anhydride modified cantharidin analogues, which are the subject of this invention, may possess one or more of the properties of being potent, selective, oxidatively stable, and cell permeable inhibitors of protein phosphatases 1 and 2A.

Therefore, according to the first aspect of this invention there are provided cell permeable inhibitors of protein phosphatases 1 and 2A, said inhibitors being anhydride modified cantharidin analogues.

According to a particular embodiment of the first aspect of this invention there are provided compounds of the formula: 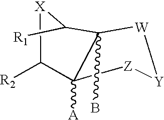
wherein R₁ and R₂ are H, aryl or alkyl; X is O, N or S; Y is O, S, SR, NH, NR, CH₂OH, CH₂OR; R is alkyl or aryl; A and B are H or CH₃; W and Z are CHOH or C=0 and R₁ and R₂ can cyclise to form a ring as follows: 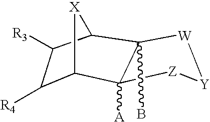
wherein R₃ and R₄ are H, aryl or alkyl

The aryl group may suitably be phenyl or naphthyl for example, and may be attached via a carbon spacer of between 6 and 10 carbon atoms. The alkyl group may suitably be C₁-C₁₀.

According to the second aspect of this invention there is provided a process for producing anhydride modified cantharidin analogues. The process may include the steps of:
dissolving a diene in a suitable solvent and adding to the resultant solution an ene.

According to a third aspect of the invention there is provided a process for producing anhydride modified cantharidin analogues, involving the step of reacting a diene with an ene.

The process may further involve hydrogenation of the adduct of the diene and ene and/or optionally, ring opening of the adduct.

Generally, the reaction conditions for the production of the anhydride modified cantharidin analogues are dependent on the aromaticity of the starting diene. Suitable reaction conditions are exemplified below.

According to a fourth aspect of this invention there is provided a method of treating a cancer which method comprises administering to a patient in need of such treatment, an effective amount of an anhydride modified cantharidin analogue of the first aspect of this invention, together with a pharmaceutically acceptable carrier, diluent and/or excipient.

The method may be carried out in conjunction with one or more further treatments for treating the cancer.

According to a fifth aspect of this invention there is provided a method of sensitising cancer cells to at least one method of treating cancer, which method of sensitising comprises administering to a patient in need of such treatment, an effective amount of an anhydride modified cantharidin analogue of the first aspect of this invention, together with a pharmaceutically acceptable carrier, diluent and/or excipient.

According to a sixth aspect of the invention there is provided a method of treating cancer which method comprises:
administering to a patient in need of such treatment, an effective amount of an anhydride modified cantharidin analogue to sensitise cancer cells of the patient to one or more cancer treatments; and utilising the one or more cancer treatments.

According to a seventh aspect of this invention there is provided a method of screening a compound for anti-cancer activity.

According to an eighth aspect of this invention there is provided a method of screening compounds for use in the fourth aspect of this invention, said method comprising screening for anti-cancer activity; and screening for ability to abrogate either the G₁ or the G₂ checkpoint of the cancer cell cycle. The method may also comprise screening for the ability of said compounds to sensitise cancer cells to one or more cancer treatments.

The one or more cancer treatments mentioned above may be selected from treatments involving cisplatin, irradiation, taxanes and antimetabolites.

The invention will hereinafter be described with reference to Examples and the accompanying figures.

### BRIEF DESCRIPTION OF THE FIGURES

FIG. 1 is a schematic representation of the structure activity data generated for inhibition by PP2A by cantharidin analogues;

FIG. 2: New cantharidin analogues.

FIG. 3: Cytotoxicity of cantharidin and the new cantharidin analogues.

FIG. 4: Cell cycle analysis 12 h following exposure to cantharidin, MK-2 or MK-4.

FIG. 5: Cell cycle analysis 18 h after 6 Gy of radiation and 12 h after exposure to cantharidin, MK-2 or MK4.

FIG. 6 (a-c): Combination index versus fraction affected: HCT116 colon cells in simultaneous combination with cisplatin and MK4.

FIG. 7 (a-b): Combination index versus fraction affected: HT29 colon cells in simultaneous combination with cisplatin and MK4.

FIG. 8 (a-c): Combination index versus fraction affected: HCT116 colon cells in simultaneous combination with taxotere and MK-4

FIG. 9 (a-c): Combination index versus fraction affected: HT29 colon cells in simultaneous combination with taxotere and MK-4.

### BEST AND OTHER MODES FOR CARRYING OUT THE INVENTION

As mentioned above, the reaction conditions for producing anhydride modified cantharidin analogues encompassed by the present invention generally depend on the aromaticity of the starting diene. This is illustrated by a description of examples of the methods wherein the starting materials are furan (Method 1 below); thiophene (Method 2 below); and pyrrole (Method 3 below).

Method 1: Furan as the Starting Diene

A solution of furan (5 equivalents) is dissolved in a suitable solvent (about 5 times the volume of furan, the solvent can be for example, ether (for room temperature reactions); or benzene or xylene (the latter two for reactions at 80 and 130° C. respectively). To this solution is added one equivalent of the ene. The reaction is then heated (or stirred at room temperature), typically for 24 hours (2 days in the case of the room temperature reaction). Upon cooling (or standing at room temperature) a precipitate forms and is collected by vacuum filtration. The adduct is then purified by recrystalisation from for example, chloroform or ethanol. In the case of the furan+maleic anhydride compound care is exercised to minimise heating as this causes a reto-Diels-Alder reaction yielding only the starting materials.

Method 2: Thiophene as the Starting Diene

Thiophene (1.016 g, 0.012 mol) and maleic anhydride (0.558.0.006 mol) are mixed at room temperature in 2.5 mL of distilled dichloromethane. This mixture is then placed inside a high pressure reactor. They are compressed to a pressure of 17 kbar at 40° C. for a period of 71 hours, after which the pressure is released and the product purified by chromatography.

Method 3: Pyrrole as the Starting Diene

To [Os(NH₃)₅OsO₂CF₃)] (CF₃SO₃)₂, (0.3511 g, 0.4 mmol) and activated magnesium (0.1511 g), pyrrole (0.45 mL, 0.6 mmol), DME (1 mL) and DMAc (0.3 mL) are added in that order. The mixture is stirred for 1 hour, the temperature gradually rising to 40° C. and then dropping. The brown slurry is filtered through a thin pad of celite, and the cake washed with DME in small portions (4×2 mL). The filtrate is added to dichloromethane (15 mL). Vigorous stirring results in the formation of yellow coloured precipitate which is collected by vacuum filtration, followed by an ether wash (2×2.5 mL). The product is dried under a stream of nitrogen yielding a yellow-tan solid (0.343 g, 84%). To this pyrrole complex is added maleimide (0.05 g, 0.515 mmol) (or any other “ene”, eg maleic anhydride, dimethyl maleate, etc) in acetonitrile. The mixture is allowed to stir at room temperature for 60 min. after which the solvent is removed by vacuum, yielding the exo isomer (0.359 g, 64%). The crude material is purified by ion-exchange column (Sephadex-CM C-25, 2×10 cm), using NaCl as the mobile phase. The complexes are precipitated by the addition of a saturated sodium tetraphenylborate solution.

The types of cancer which are amenable to treatment by these compounds include those types of cancer which are inherently resistant to conventional chemotherapy. Typically, these types of cancer are represented by the more common and more aggressive tumour types such as, but not limited to, colon cancer and non small-cell lung cancer.

The compounds of this invention are suitably administered intravenously, although other modes of administration are possible. Pharmaceutically acceptable diluents, adjuvants, carriers and/or excipients may be used in conjunction with the compounds of this invention.

Suitable such pharmaceutically acceptable substances are those within the knowledge of the skilled person and include compounds, materials and compositions deemed appropriate.

Actual dosage levels of the compounds of the invention may be varied so as to obtain an amount of the active ingredient which is effective to achieve the desired response for a particular patient, composition and mode of administration.

The dosage level can be readily determined by the physician in accordance with conventional practices and will depend upon a variety of factors including the activity of the particular compound of the invention to the administered, the route of administration, the time of administration, the rate of excretion of the particular compound employed, the age, sex, weight, condition, general health and prior medical history of the patient being treated, and like factors well known in the medical arts.

The compounds of this invention may also sensitise cancer cells to other methods of treatment. For example, typically these methods include irradiation and treatment with platinum anti-cancer agents, for example cisplatin.

In addition, sensitisation may also be brought about by, for example the use of the plant alkaloids vinblastine and vincristine, both of which interfere with tubulin and the formation of the mitotic spindle, as well as taxanes and antimetabolites, including 5-fluorouracil, methotrexate and antifolates.

In particular, the compounds of this invention sensitise those cells with deficient p53 activity.

When screening for anti-cancer activity as contemplated by the invention, various cancer cell lines may be chosen. These are typically both haematopoietic and solid tumour cell lines with varying p53 status and include: L1210 (murine leukaemia, p53 wildtype), HL60 (human leukaemia, p53 nul), A2780 (human ovarian carcinoma, p53 wildtype), ADDP (cisplatin resistant A2780 cells, p53 mutant), SW480 (human colon carcinoma, p53 mutant), WiDr (human colon carcinoma, p53 mutant), HT29 (human colon carcinoma, p53 mutant), HCT116 (human colon carcinoma, p53 wildtype) and 143B (human osteosarcoma, p53 mutant).

In addition to the methods for screening for anti-cancer activity, the following procedures may be suitably used in the remainder of the screening process. For example, when screening for the ability to abrogate the G₁ and/or the G₂ checkpoint of the cancer cell cycle, the following are suitably used:

Cell Cycle Method

The cells are fixed in 70% ethanol and stored at −20° C. until analysis is performed (1-2 weeks). After fixing, the cells are pelleted and incubated in PBS containing propidium iodide (40 mg/ml) and RNase A (200 mg/ml) for at least 30 min at room temperature. The samples (2×10⁴ events) are analysed using a Becton Dickson FACScan, fluorescence is collected in fluorescence detector 2 (FL2), filter 575/30 nm band pass. Cell cycle distribution is assessed using Cell Quest software (Becton Dickson).

Those protein phosphatase inhibitors which show abrogation of either the G₁ or G₂ checkpoint will then be exploited in combination studies with either radiation exposure or chemotherapy drugs incubation. The MTT (3-[4,5-dimethylthiazol-2-yl]2,5-diphenyl-tetrazolium bromide) assay is used to determine whether a synergistic, antagonistic or additive effect is induced. The Median Effect method is adopted to mathematically determine the optimal combination index of the treatments chosen (Chou and Talalay, 1984). This method has been extensively used to investigate the cytotoxicity of various drug combinations including cisplatin and D1694 (Ackland et al 1996; 1998). A combination index value less than 1 indicates synergism, a value equal to 1 indicates additivity and a value greater than one indicates antagonism.

Cytotoxicity Assay

When screening for the ability to sensitise cancer cells to conventional chemotherapy and irradiation, the following methods are suitably used:

Cells in a subconfluent phase are transferred to 96-well microtitre plates. L1210 cells are plated at a density of 1000 cells/well in 100 μl medium, while all other cell lines are plated at a density of 2000-25000 cells/well. The cells are left for 24 h prior to treatment to ensure exponential growth has been achieved, 24 h after plating (day 0), 100 μl of phosphatase inhibitor is added to each well, control wells received 100 μl of medium only. Drug exposure time is 72 h (day 3). The effect of phosphatase inhibition is tested in triplicate over a concentration range of 1×10¹³M−1×10⁻⁸ M. Growth inhibitory effects are evaluated using the MTT assay and absorbance read at 540 mm. The IC₅₀ is the drug concentration at which cell growth is 50% inhibited based on the difference of optical density on day 0 and day 3 of drug exposure. Cytotoxicity is evaluated using a spectrophotometric assay which determines the percentage of cell growth following exposure of the cells to various concentrations of the phosphatase inhibitors for a period of 72 hours. The subsequent dose response curve is used to calculate IC₅₀ values (the drug concentration at which cell growth is 50% inhibited).

Most drug discovery has focused on the development of new single agents. However, in light of the success of combination chemotherapy it is increasingly apparent that successful anticancer treatment of the future will be based upon the discovery of agents which are synergistic in their action. In view of this, the cytotoxicity of phosphatase inhibitors in combination with either radiation, cisplatin, taxanes, antimetabolites or plant alkaloids is examined. As indicated above, calyculin which by itself is not cytotoxic, enhances irradiation induced cell death. Similarly abrogation of the G₂ checkpoint by either, caffeine or UCN-01, also enhances the cytotoxicity of y irradiation in cells with mutant p53 (CA46 and HT-29 cells) (Powell et al., 1995; Russell et al. 1995: Wang et al., 1996). DNA damage induced by irradiation causes both a G₁ and G₂ cell cycle arrest. In p53 mutant cells, the G₁ checkpoint is absent. However, following irradiation the cells will still arrest in the G₂ phase, and potentially repair the damage. P53 mutant cells are generally more resistant to conventional chemotherapy and produce more aggressive tumours. Therefore, in p53 deficient cells, DNA damage that is not detected by the G₁ checkpoint will be picked up by the G₂ checkpoint. If the cells are deficient in both of these checkpoints then it is believed that the cells will be unable to initiate repair mechanisms and will be more unstable and increasingly susceptible to cell death induced by DNA damage.

Cisplatin is another commonly used anticancer treatment which binds to DNA and produces DNA crosslinks and strand breaks. Cisplatin is particularly useful in the treatment of testicular carcinoma, small cell carcinoma of the lung, bladder cancer, and ovarian cancer. Repair of cisplatin induced DNA damage is mediated via nucleotide excision repair which is coordinated by p53 activation of Gadd45 (Smith et al., 1994). In this context, it has been suggested that cells that are p53 mutant are more sensitive to cisplatin treatment (Hawkins et al., 1996). A number of researchers have investigated this proposal in p53 mutant cell lines and in p53 mutant tumours, with mixed results. While it is apparent that cisplatin is more cytotoxic in cells lines that are deficient in p53 (induced via papillomavirus) compared to the p53 proficient cells (Hawkins et al., 1996), it is harder to test this hypothesis in tumours and in cisplatin resistant cells as they may have several undefined mutations in their genome which would confound such studies (Herod et al., 1996). Nevertheless, the G₂ abrogator UCN-01 (7-hydroxystaurosporine, a protein kinase inhibitor) has been shown to markedly enhanced the cell-killing activity of cisplatin in MCF-7 cells defective for p53 function (Wang et al., 1996).

The development of chemotherapeutic agents which selectively target p53 mutant cells is desirable since 50% of tumours have either a mutated or deleted p53 gene. Many of these p53 deficient cells and tumours are inherently resistant to conventional chemotherapy and represent the common more aggressive tumour types such as colon cancer, and non-small cell lung cancer. Thymidylate synthase (TS) inhibitors are another class of commonly used anticancer agents. TS catalyses a critical step in the pathway of DNA synthesis by converting dUMP to dTMP by methylation using the co-substrate N5,N10-methylene tetrahydrofolate (CH₂-THF) as a methyl donor. This step is the only de novo source of dTMP, which is subsequently metabolised to dTTP exclusively for incorporation into DNA during synthesis and repair (Jackman & Calvert, 1995). Thus. TS is a key regulatory enzyme during the S-phase of the cell cycle. Lack of dTTP results in DNA damage and ultimately cell death, but the process(es) by which cell death occurs is not clear. TS inhibitors such as fluorouracil, raltitrexed, and LY231514 play a pivotal role in anticancer treatment and are often the first line treatment of many cancers (Peters & Ackland, 1996). We propose that the TS inhibitor Thymitaq (Zarix, Ltd) be used in combination with cantharidin analogues. Thymitaq is a direct and specific TS inhibitor which does not require active transport into the cell nor does it require intracellular activation for its action.

The following examples are not to be construed as limiting on the scope of the invention as indicated above.

### EXAMPLE 1

Chemistry

Anhydride modified cantharidin analogues were synthesised by a variety of modified literature procedures, as set out in schemes 1 and 2. These modifications are embodied in the three methods, which depend on the aromaticity of the starting dienes, set out above. The dimethyl ester (3), which was prepared by the application of high pressure, 17 kbar, 40° C., 61 hours, as shown in scheme 3. 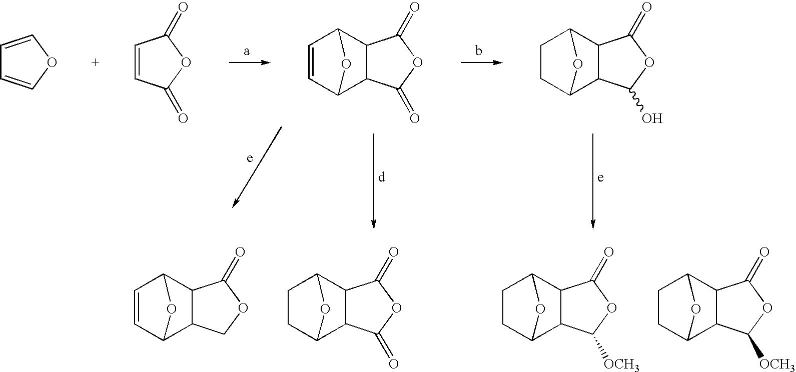
Scheme 1.a. Furan:maleic anhydride (5:1), diethylether, 2d, RT, 96%; b. H₂/10% Pd—C/EtOH; c. p-TosOH, MeOH, chromatography; d. H₂/10% Pd—C/Acetone; e. NaBH₄ then HCl. 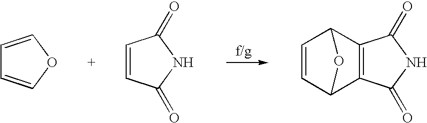
Scheme 2. Reagents and Conditions: f. Furan:maleimide (5:1), diethyl ether, 7d, in dark, 75%, exo product; g. Furan:Maleimide (5:1), diethylether, sealed tube 12 h, 90° C., 66%, endo product. 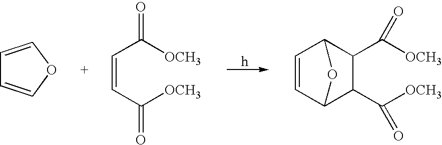
Scheme 3. Reagents and Conditions: h.
Furan:dimethylmaleate (2:1), CH₂Cl₂, 17 Kbar, 40° C. 61 h, 56%.

### EXAMPLE 2

Development of Potent, Selective, Oxidatively Stable, and Cell Permeable Inhibitors of Protein Phosphatases 1 and 2A.

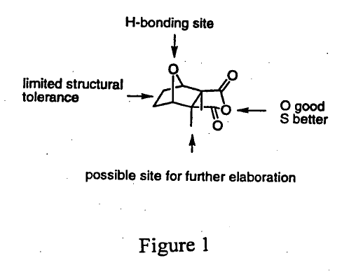

Crude natural product extracts have yielded isopalinurin and a series of cantharidin analogues have been synthesised. In this context, the present inventors have developed the simple cantharidin analogue which is PP1 selective (IC₅₀=50 mM, with 0% inhibition of PP2A at concentrations ≧1000 mM) representing the first small molecule to exhibit selectivity for PP1. Results have indicated that a series of simple synthetic modification of the cantharidin skeleton also allows the synthesis of a PP2A selective compound (see FIG. 1).

The present inventors have previously demonstrated that a facile ring opening of an anhydride is crucial to inhibition of PP2A. This is not possible with c (previous studies with the 7-0, and this analogue indicated considerable hydrolytic stability of the maleimide link). It is also interesting to note that endothal thioanhydride is three fold more potent than cantharidin, with the S atom being an important factor. It is thus envisaged that the 7-S group presents itself to the active sites metals and the N—H of the maleimide occupies the hydrogen bond cavity normally reserved for the 7-0 substituent cantharidin.

Structure of Cantharidin and Selective Analogues 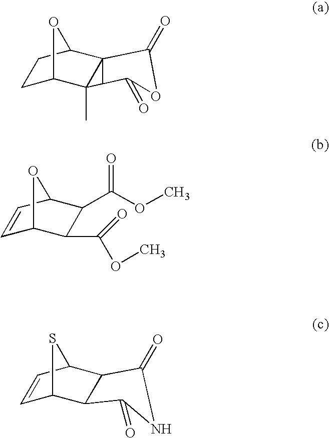
(a) Shows structure of cantharidin;
(b) Shows PP1 selective analogue; and
(c) Shows PP2A selective analogue. In the case of panel (c) IC₅₀˜25 mM.

On the basis of these results and previous experience in our laboratory (synthesis and molecular modelling of cantharidin inhibitors at PP1 and PP2A), we have designed a series of analogues which are more active and selective, whilst retaining the desirable properties of stability and cell permeability.

The synthetic pathways to these analogues are shown in schemes 1-3. Each scheme allows for modification of the basic skeleton, and in some cases the insertion of beneficial feature that were present in the more complex natural toxin(s) (eg okadaic acid, calyculin, microcystin, etc). The inclusion of these features is designed to provide enhanced selectivity and potency.

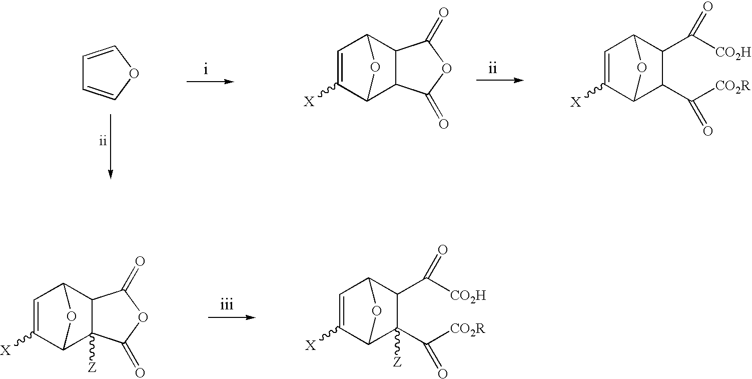

### EXAMPLE 3

Synthetic Development of a Series of PP1 and PP2A Analogues of Cantharidin.

(i) Diels-Alder addition (maleic anhydride) and subsequent manipulations of X; (ii) Diels-Alder addition (substituted maleic anhydrides), introduction and manipulation of Z (Z=hydrophobic tail; eg long chain nitrile: cf Calyculin A, long chain terminating in a Spiro acetal: cf Tautomycin. Okadaic acid; long chain terminating in an aromatic ring: cf Adda in Microcystin-LR; (iii) stereospecific ring opening of the anhydride allowing further manipulations of the newly released functional groups (see scheme 2).

In this instance we have developed synthetic protocols in our laboratory that allow the facile assembly of these analogues. Biological evaluation and molecular modelling of the most active molecules will allow compounds to be evaluated.

Additional modification to the basic structure can be obtained as exemplified below.

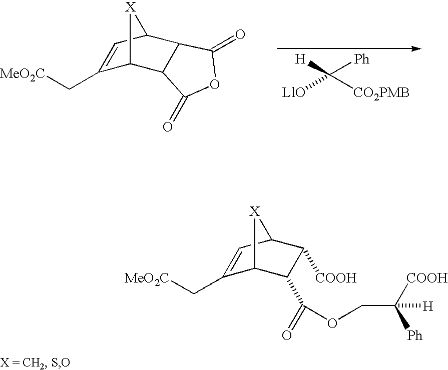

### EXAMPLE 4

A specific example of one class of cantharidin analogue that shows promise as a selective inhibitor of protein phosphatases 1 and 2A.

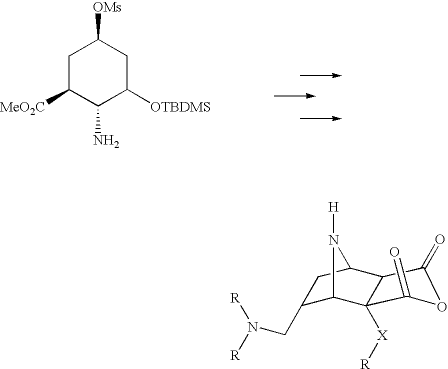

### EXAMPLE 5

Stereospecific Route Towards 7-azabicyclo[2.2.1]heptanes

We have shown that the introduction of the bridgehead nitrogen improves the potency, selectivity and stability of similar analogues, the above pathway has been developed to further improve the bio-activity of these analogues. The synthetic routes alluded to herein may allow the rapid assembly of the target molecules.

Those agents which meet the requirements of being stable, specific, potent, and membrane permeable protein phosphatase inhibitors are screened for their anti-cancer activity.

### EXAMPLE 6

Biochemistry

All synthesised compounds were tested for their ability to inhibit protein phosphatases 1 and 2A. Initial investigations were carried out at 100 mM. Promising analogues were then assayed in triplicate for estimation of IC₅₀ values.

Protein phosphatase 1 and 2A were partially purified from chicken skeletal muscle essentially as described by Cohen Protein phosphatase activity was measured at 37° C. in 50 mM Tris-HCl buffer (pH 7.4), 0.1 mM EDTA, 5 mM caffeine, 0.1% 2-mercaptoethanol and 1 mg/ml bovine serum albumin using 30 mg [³²P]-phosphorylase as substrate. The total assay volume was 30 ml. The assay conditions were restricted to 20% dephosphorylation to ensure linearity and inhibition of protein phosphatase activity was determined by including cantharidin or its analogues at the required concentrations in the reaction buffer. Reactions were terminated by the addition of 0.1 ml ice cold 20% trichloroacetic acid. Precipitated protein was pelleted by centrifugation and the radioactivity in the supernatant measured by liquid scintillation counting. Data is expressed as the percentage inhibition with respect to a control (absence of a competing compound) incubation.

### EXAMPLE 7

Screening Various PP1 and PP2A Inhibitors for Anti-Cancer Activity

(a) Cytotoxicity of Protein Phosphatase Inhibition:

Those PP1 and PP2A inhibitors which fulfil the requirements detailed above were tested in various cancer cell lines. The cells lines chosen for study included both haematopoietic and solid tumour cell lines with varying p53 status and include:

| | |
|---|---|
| L1210 | (murine leukaemia, p53 wildtype), |
| HL60 | (human leukaemia, p53 nul), |
| A2780 | (human ovarian carcinoma, p53 wildtype), |
| ADDP | (cisplatin resistant A2780 cells, p53 mutant), |
| SW480 | (human colon carcinoma, p53 mutant), |
| WiDr | (human colon carcinoma, p53 mutant). |
| HT29 | (human colon carcinoma, p53 mutant) |
| HCT116 | (human colon carcinoma, p53 wildtype) |
| 143B | (human osteosarcoma, p53 mutant) |

Anti-cancer screening of the protein phosphatase inhibitors is assessed using the MTT assay. This assay determines cell viability by the ability of mitochondrial dehydrogenase to produce formazan crystals from 3-(4,5-dimethylthiazol-2-yl)-2,5-diphenyltetrazolium bromide. The viable cell number/well is directly proportional to the production of formazan, which following solubilization, can be measured spectrophotometrically (540 nm). This technique is also used by the National Cancer Institute to screen for new anticancer agents.

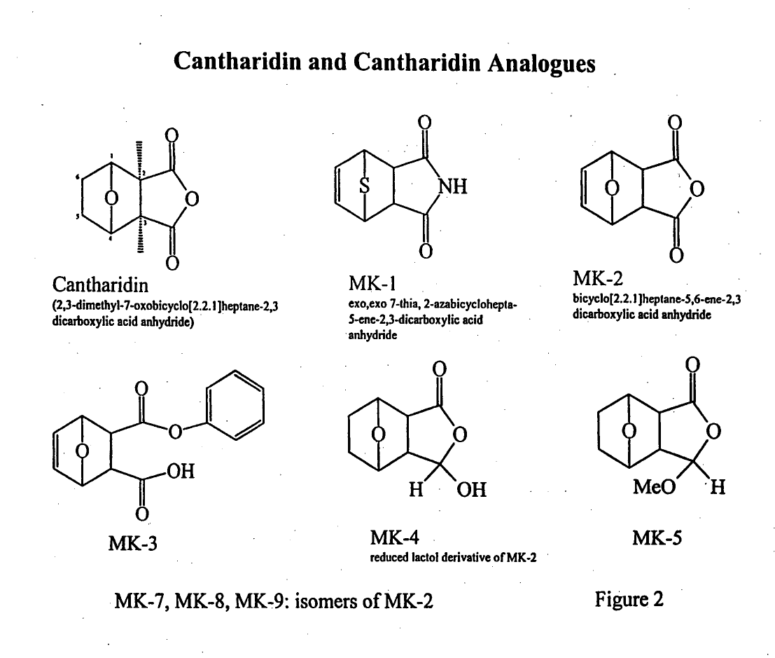

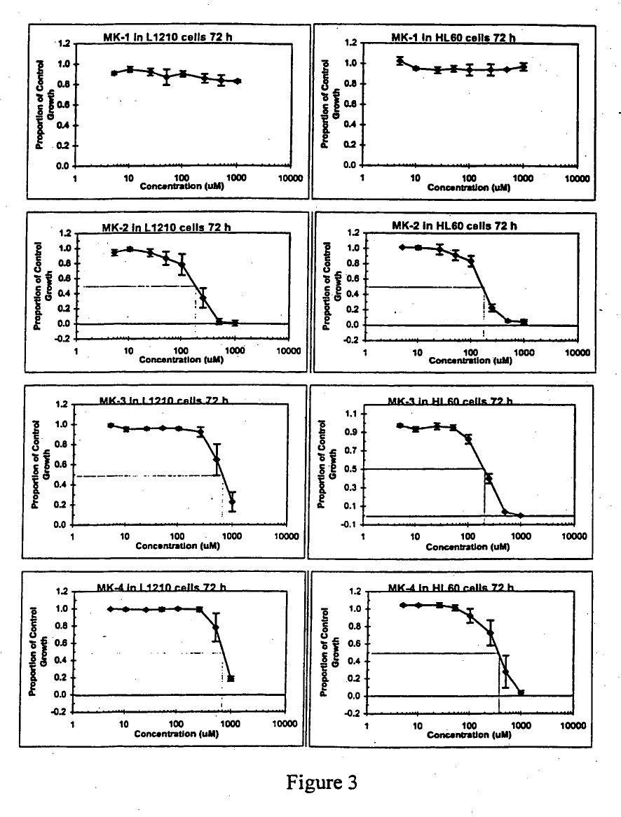

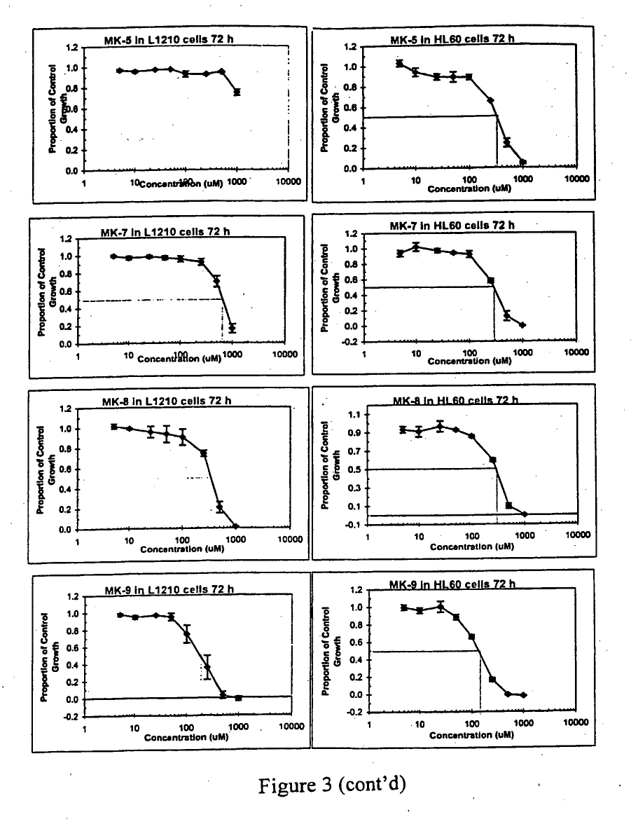

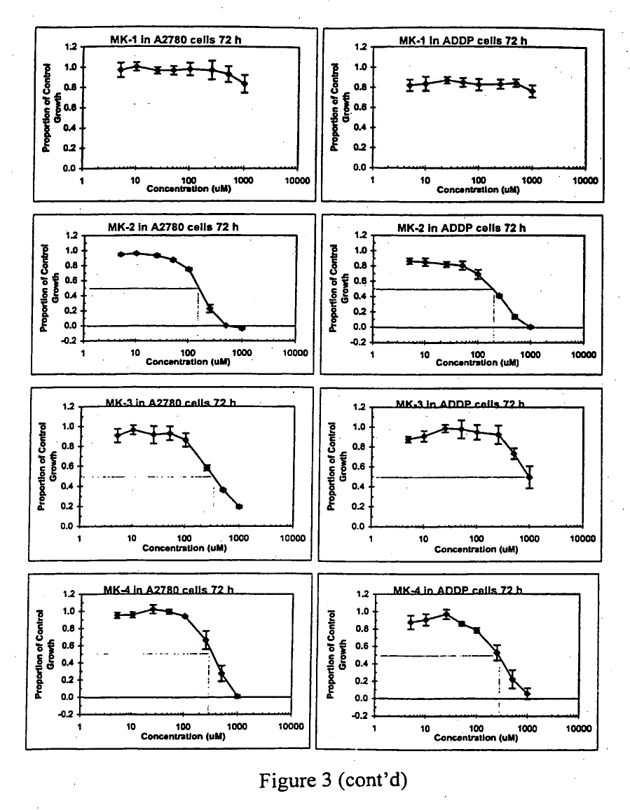

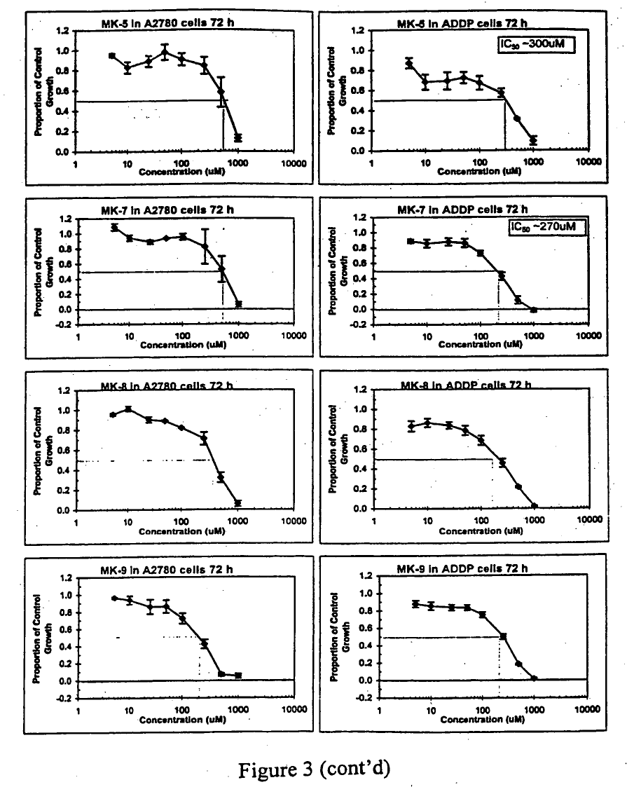

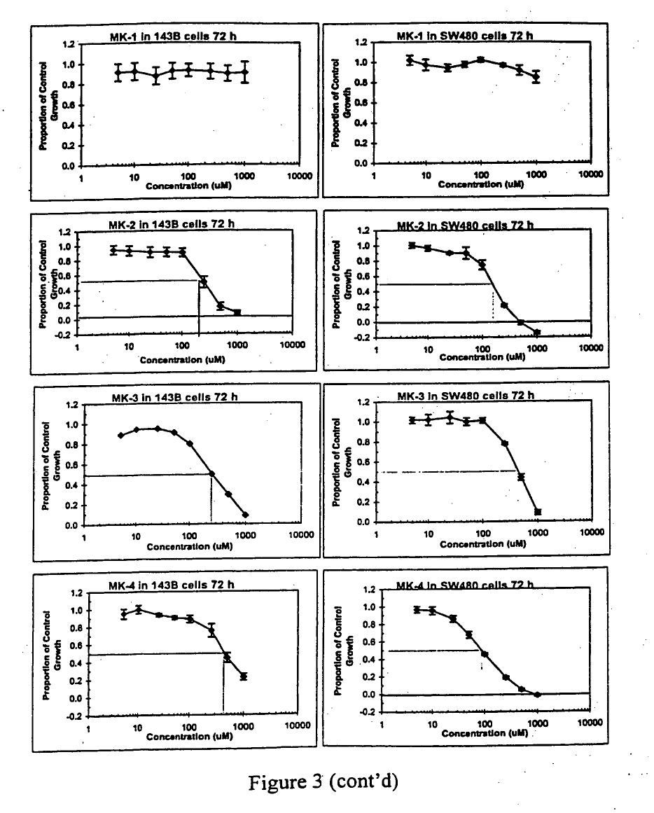

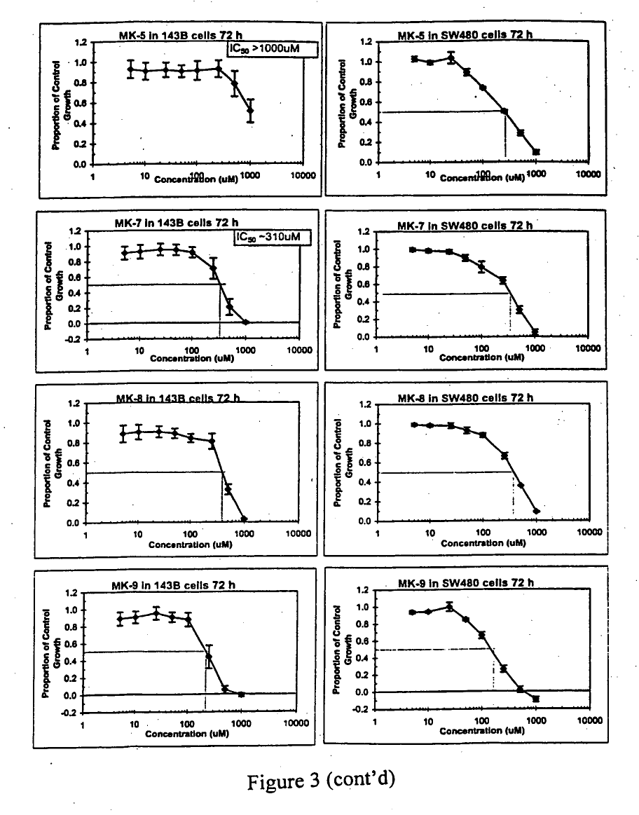

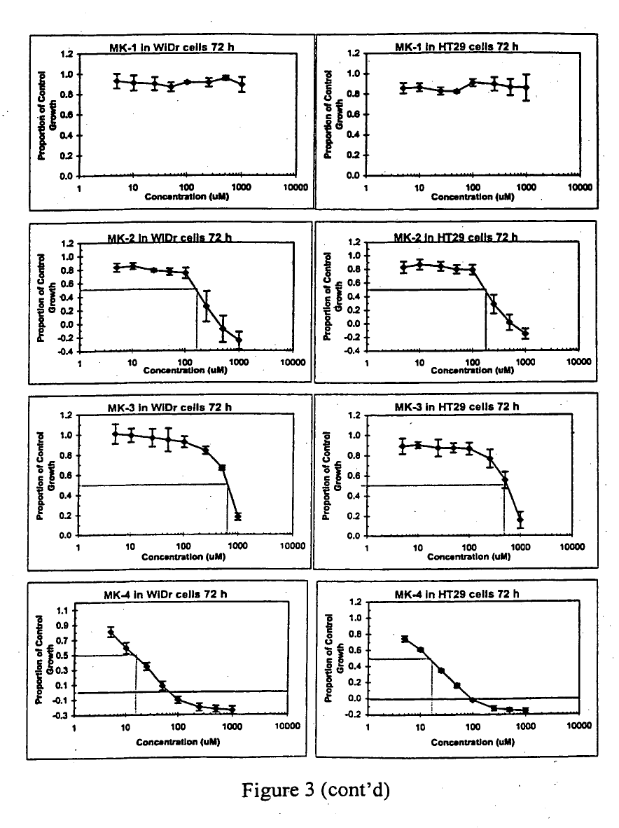

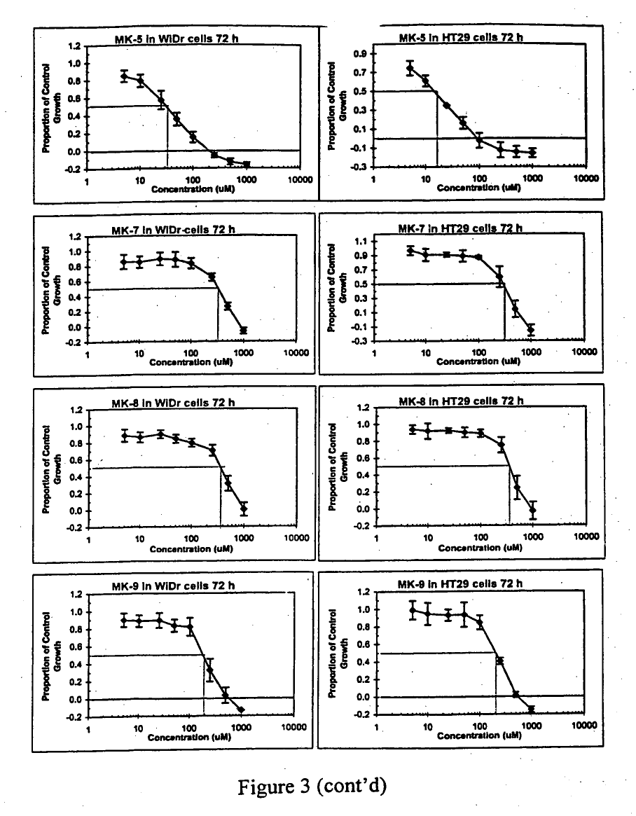

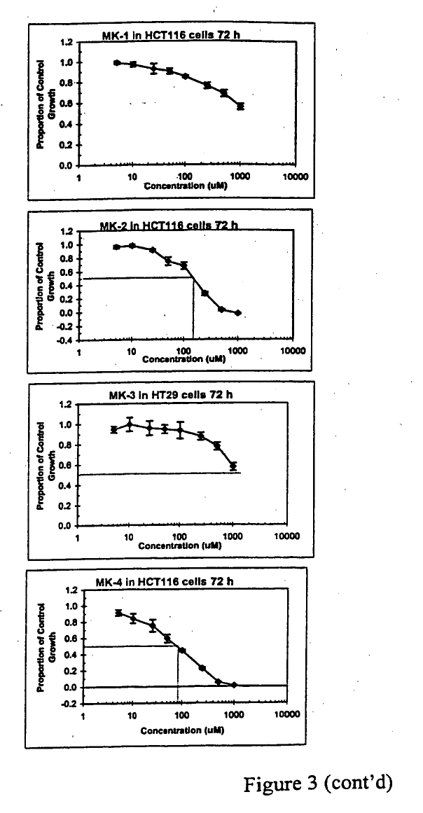

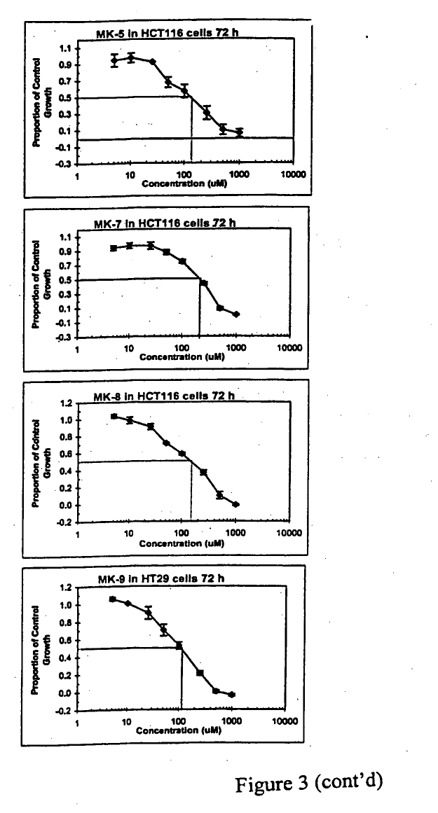

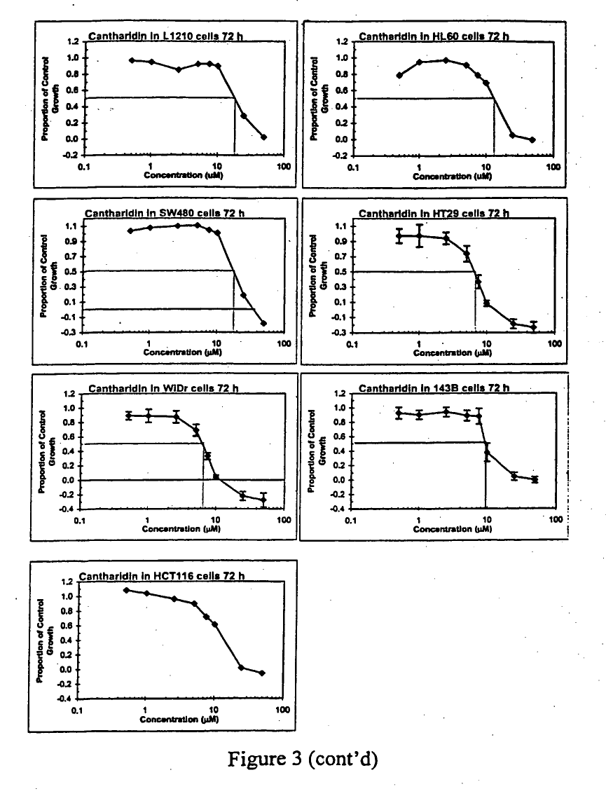

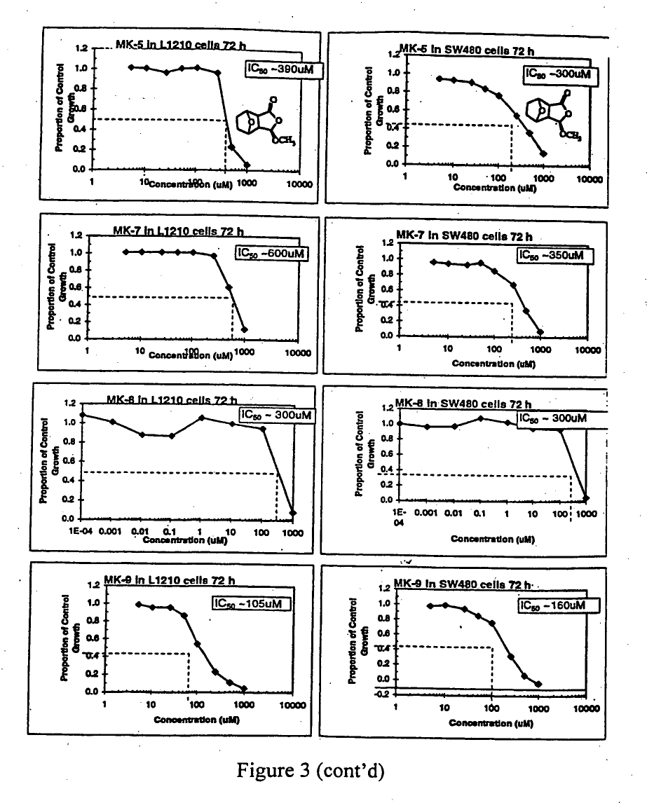

As described herein a number of cantharidin analogues have been synthesised and tested for their anticancer activity in nine cancer cell lines using the MTT assay after 72 h exposure. These new analogues are shown in FIG. 2 and have been designated MK-1 through to MK-9. The cytotoxicity (IC₅₀) of these cantharidin analogues is shown in Table 1 and FIG. 3. In summary, the MK-1 analogue did not show any significant cytotoxicity in any of the cell lines tested (IC₅₀>1000 μM). Only marginal cytotoxicity across all cell lines tested was observed for MK-3 (IC₅₀ 247 to >1000 μM), MK-7 (IC₅₀

TABLE 1
IC₅₀ values of tumour cell lines after 72 h continuous exposure to cantharidin and cantharidin analogues.

| Tumour | Cell | p53 | IC₅₀ (mean ± SE) after 72 h continuous exposure (μM) | | | | | | | | |
|---|---|---|---|---|---|---|---|---|---|---|---|
| type | line | status | Cantharidin | MK-1 | MK-2 | MK-3 | MK-4 | MK-5 | MK-7 | MK-8 | MK-9 |
| Murine | L1210 | wt | 18± | >1000 | 185 ± 51 | 647 ± 132 | 680 ± 97 | >1000 | 367 ± 37 | 337 ± 19 | 192 ± 56 |
| Leukae- | | | | | | | | | | | |
| mia | | | | | | | | | | | |
| Human | HL60 | nul | 13± | >1000 | 177 ± 3 | 247 ± 55 | 393 ± 103 | 323 ± 13 | 293 ± 7 | 297 ± 3 | 133 ± 9 |
| Leukae- | | | | | | | | | | | |
| mia | | | | | | | | | | | |
| Human | A2780 | wt | ± | >1000 | 157 ± 9 | 317 ± 17 | 333 ± 55 | 567 ± 109 | 357 ± 102 | 313 ± 61 | 187 ± 9 |
| Ovarian | | | | | | | | | | | |
| Human | ADDP | mt | 12 ± 0.8 | >1000 | 183 ± 17 | >1000 | 275 ± 56 | 260 ± 40 | 210 ± 18 | 208 ± 19 | 233 ± 23 |
| Ovarian | | | | | | | | | | | |
| Human | 143B | mt | 10.2 ± 1.2 | >1000 | 248 ± 29 | 665 ± 225 | 450 ± 50 | >1000 | 327 ± 67 | 385 ± 43 | 223 ± 44 |
| Osteo- | | | | | | | | | | | |
| sarcoma | | | | | | | | | | | |
| Human | HCT116 | wt | 12± | >1000 | 160 ± 10 | >1000 | 78 ± 7 | 143 ± 23 | 180 ± 20 | 173 ± 22 | 107 ± 12 |
| Colon | | | | | | | | | | | |
| Human | HT29 | mt | 6.4 ± 0.7 | >1000 | 183 ± 20 | 530 ± 112 | 14 ± 0.3 | 28 ± 1 | 297 ± 58 | 373 ± 54 | 205 ± 13 |
| Colon | | | | | | | | | | | |
| Human | WiDr | mt | 6.1 ± 0.5 | >1000 | 198 ± 53 | 620 ± 31 | 15 ± 3 | 31 ± 10 | 320 ± 20 | 367 ± 44 | 190 ± 35 |
| Colon | | | | | | | | | | | |
| Human | SW480 | mt | 17.5± | >1000 | 155 ± 9 | 444 ± 27 | 88 ± 5 | 247 ± 14 | 333 ± 22 | 353 ± 20 | 147 ± 14 |
| Colon | | | | | | | | | | | |

wt = wildtype,
mt = mutant.

180-367 μM) and MK-8 (IC₅₀ 173-385 μM). Greater cytotoxicity was observed with MK-2 (IC₅₀ 157-248 μM) and MK-9 (IC₅₀ 107-233 μM) which was also consistent across the nine cell lines. The greatest cytotoxicity was observed with the MK-4 and MK-5 analogues, however, the magnitude of this response was cell line dependent. In this context, MK-4 and MK-5 were selectively more cytotoxic in the human colon cancer cell lines (IC₅₀ 14-88 μM; 28-247 μM) compared with leukaemia (IC₅₀ 393-680 μM; 323->1000 μM) ovarian (IC₅₀ 275-333 μM; 260-567 μM), and osteosarcoma (IC₅₀ 450 μM; >1000 μM) cells respectively.

(b) Abrogation of Cell Cycle Checkpoints:

The ability of the protein phosphatase inhibitors to abrogate the G₁ or G₂ checkpoint of the cell cycle may be determined by cell cycle analysis using flow cytometry. Briefly, asynchronous cell cultures are harvested 18 h after 6 Gy irradiation and/or 12 h incubation with the protein phosphatase inhibitor. Depending upon the p53 status of the cell line, radiation treatment alone will induce arrest in either G₁ and/or G₂ phase of the cell cycle.

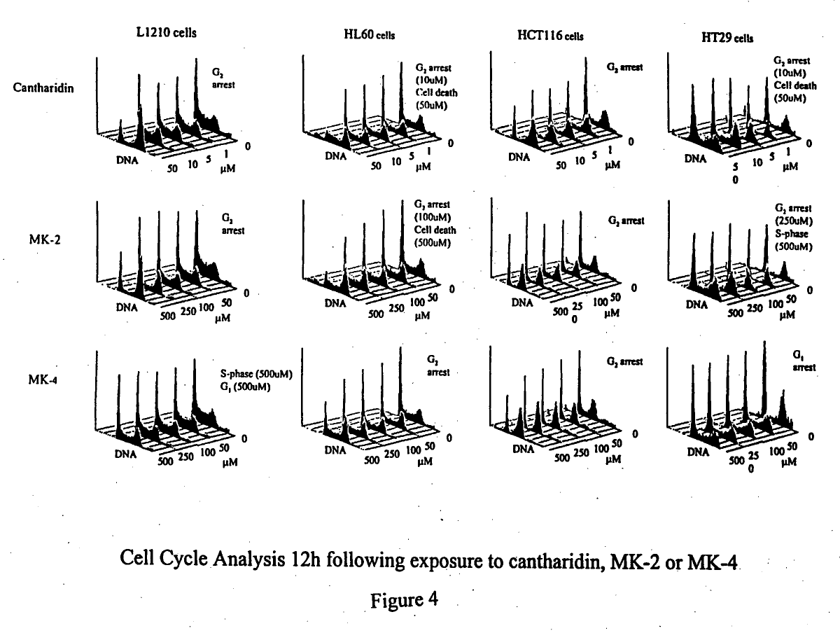

Data shown in Table 2 and FIG. 4 show the cell cycle response of L1210, HL60, HT29 and HCT116 cells to cantharidin and the new cantharidin analogues MK-2 and MK-4 after 12 h exposure. In summary, cantharidin and MK-2 produced a similar response and induced G₂ arrest in all four cell lines tested. MK-4 also induced G₂ arrest but only in L1210. HL60 and HCT116 cells. In HT29 cells, MK-2 induced G₁ cell cycle arrest. The magnitude of the cell cycle arrest induced by these drugs directly correlated with their cytotoxicity in the respective cell lines. The ability of the parent compound cantharidin to inhibit cell growth is also shown (IC₅₀ 6.1-18 μM). The cytotoxicity of the

TABLE 2
Cell Cycle Analysis
Cell Cycle Distribution (percentage of total) of tumour cell lines 12 h
after cantharidin or cantharidin analogue treatment.
Method: Flow Cytometry of Propidium Iodide stained cells.

| | | L1210 cells | | | | | HL60 cells | | | | |
|---|---|---|---|---|---|---|---|---|---|---|---|
| Agent | μM | sub G₁ | G₁ | S | G₂ + M | | sub G₁ | G₁ | S | G₂ + M | |
| Cantharidin | 0 | 0.5 | 47.4 | 34.3 | 19.4 | | 1.9 | 45.5 | 25.8 | 28.2 | |
| | 1 | 0.5 | 45.8 | 33.7 | 21.6 | | 1.5 | 44.0 | 26.1 | 29.7 | |
| | 5 | 0.6 | 46.5 | 32.6 | 21.9 | | 1.7 | 41.4 | 27.7 | 30.6 | |
| | 10 | 0.5 | 49.1 | 33.0 | 18.9 | | 1.7 | 41.8 | 27.5 | 30.4 | G₂ arrest |
| | 50 | 1.9 | 22.0 | 27.8 | 50.6 | G₂ arrest | 19.3 | 16.2 | 31.6 | 34.7 | Cell Death |
| MK-2 | 0 | 0.4 | 40.1 | 28.4 | 32.1 | | 2.1 | 45.6 | 21.0 | 32.6 | |
| | 50 | 0.3 | 42.7 | 26.2 | 31.7 | | 1.8 | 44.1 | 23.8 | 31.4 | |
| | 100 | 0.6 | 45.2 | 22.4 | 32.4 | | 1.8 | 43.3 | 23.6 | 32.4 | |
| | 250 | 2.4 | 46.7 | 14.3 | 36.7 | | 3.2 | 37.7 | 23.8 | 36.4 | G₂ arrest |
| | 500 | 3.9 | 26.3 | 10.1 | 60.0 | G₂ arrest | 18.8 | 17.8 | 21.6 | 43.1 | Cell death |
| MK-4 | 0 | 0.8 | 42.0 | 26.9 | 31.7 | | 2.3 | 49.9 | 21.6 | 27.4 | |
| | 50 | 0.5 | 42.0 | 26.9 | 32.0 | | 1.9 | 44.7 | 22.3 | 32.3 | |
| | 100 | 0.4 | 43.2 | 25.4 | 32.5 | | 2.5 | 45.3 | 22.6 | 30.6 | |
| | 250 | 0.5 | 45.7 | 24.6 | 30.5 | | 6.0 | 40.0 | 23.0 | 32.0 | |
| | 500 | 1.1 | 47.5 | 18.6 | 33.9 | Slight Δ | 6.1 | 27.8 | 22.8 | 44.4 | G₂ arrest |
| | | HCT116 cells | | | | | HT29 cells | | | | |
| Agent | μM | sub G₁ | G₁ | S | G₂ + M | | sub G₁ | G₁ | S | G₂ + M | |
| Cantharidin | 0 | 6.5 | 43.3 | 14.6 | 36.4 | | 11.1 | 45.3 | 8.0 | 36.0 | |
| | 1 | 2.2 | 39.9 | 17.2 | 41.9 | | 9.0 | 46.2 | 7.8 | 37.4 | |
| | 5 | 2.9 | 39.9 | 16.8 | 41.8 | | 4.0 | 47.4 | 9.3 | 39.8 | |
| | 10 | 6.2 | 38.0 | 14.9 | 42.0 | | 2.8 | 42.7 | 14.6 | 40.3 | G₂ arrest |
| | 50 | 11.1 | 25.1 | 17.8 | 48.1 | G₂ arrest | 15.1 | 46.0 | 14.7 | 26.0 | Cell Death |
| MK-2 | 0 | 4.7 | 44.2 | 13.7 | 36.8 | | 6.0 | 46.4 | 9.3 | 37.5 | |
| | 50 | 1.3 | 47.2 | 13.8 | 37.4 | | 9.4 | 45.3 | 7.6 | 37.1 | |
| | 100 | 1.7 | 47.2 | 16.0 | 34.5 | | 3.6 | 49.8 | 8.2 | 37.6 | |
| | 250 | 1.4 | 52.8 | 11.1 | 34.3 | | 4.2 | 41.4 | 11.2 | 42.5 | G₂ arrest |
| | 500 | 2.5 | 39.4 | 11.3 | 46.5 | G₂ arrest | 5.2 | 44.5 | 15.4 | 33.6 | S-phase↑ |
| MK-4 | 0 | 4.1 | 44.0 | 12.5 | 39.4 | | 5.5 | 45.7 | 7.4 | 41.4 | |
| | 50 | 4.5 | 43.9 | 11.4 | 40.7 | | 4.7 | 51.4 | 12.3 | 31.6 | |
| | 100 | 2.0 | 41.4 | 13.6 | 44.1 | | 6.0 | 52.3 | 12.5 | 29.4 | |
| | 250 | 3.9 | 36.2 | 14.1 | 46.9 | | 7.0 | 53.2 | 11.9 | 27.6 | |
| | 500 | 9.6 | 29.0 | 15.7 | 46.5 | G₂ arrest | 3.4 | 53.7 | 14.1 | 29.1 | G₁ arrest |

cantharidin is greater than for its analogues. Interestingly, cantharidin also showed slight selectivity towards the colon cancer cells.

If the protein phosphatase inhibitor abrogates the G₂ checkpoint then the cells will not arrest in the G₂ phase of the cell cycle and the cells will continue through the cell cycle and accumulate in the G₁ phase of the cell cycle only. Similarily if the protein phosphatase inhibitors abrogates the G₁ checkpoint then the cells will not arrest in the G₁ phase of the cell cycle and accumulate in the G₂ phase of the cell cycle only. Cell cycle analysis using propidium iodide labelling of DNA has been used extensively in our laboratory to assess the effect of specific anticancer agents that induce S-phase cell cycle arrest and apoptotic cell death (Sakoff, Ackland and Stewart, 1998). Experiments were performed on a Becton Dickinson FACScan and using Cell Quest software.

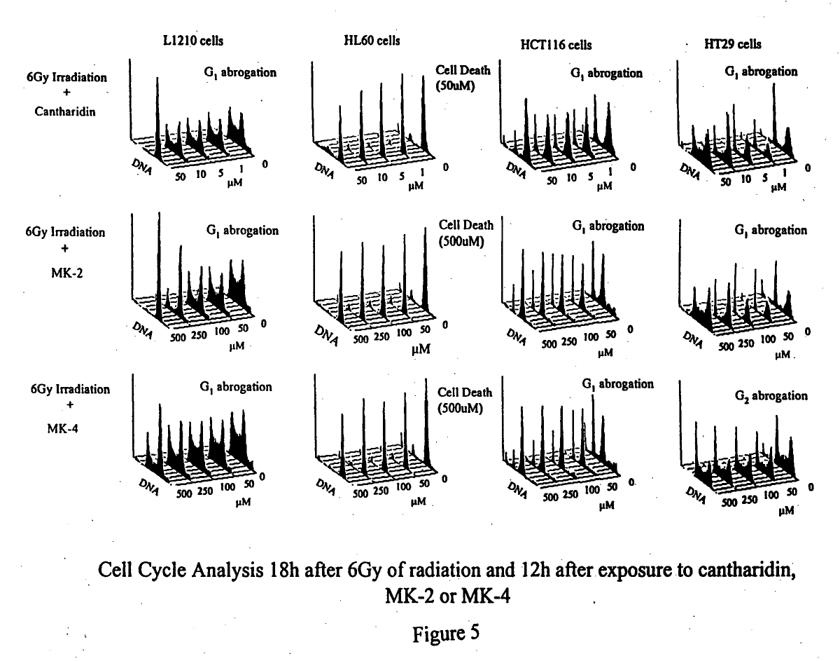

Data shown in Table 3 and FIG. 5 show the cell cycle response of L1210, HL60, HT29 and HCT116 cells. The cells were treated with 6 Gy of radiation and then treated with cantharidin 6 h later. The ability to abrogate cell cycle arrest was assessed is 12 h after the addition of the drugs. Cantharidin and MK-2 both abrogated radiation induced G₁ arrest in all cell lines. MK4 also abrogated G₁ arrest in L1210, HL60 and HCT116 cells. In HT29 cells, MK-4 induced abrogation of the G₂ checkpoint. It is important to note that the exposure of HT29 cells to MK-4 induced the greatest cytotoxicity (IC₅₀ 14 μM) as determined by the MTT assay. Not surprisingly, the ability to abrogate the G₂ checkpoint was more lethal than the ability to abrogate the G₁ checkpoint.

TABLE 3
Checkpoint Abrogation
Cell Cycle Distribution (percentage of total) of tumour cell lines 18 h after 6Gy of radiation and 12 h after
cantharidin or cantharidin analogue treatment.
Method: Flow Cytometry of Propidium Iodide stained cells.

| | | L1210 cells | | | | | HL60 cells | | | | |
|---|---|---|---|---|---|---|---|---|---|---|---|
| Agent | μM | sub G₁ | G₁ | S | G₂ + M | | sub G | G₁ | S | G₂ + M | |
| Cantharidin | 0 | 1.6 | 25.3 | 35.8 | 38.8 | | 6.6 | 5.3 | 3.2 | 85.3 | |
| | 1 | 1.6 | 27.2 | 25.6 | 37.5 | | 6.2 | 5.2 | 3.0 | 85.8 | |
| | 5 | 2.4 | 25.8 | 31.9 | 41.5 | | 4.4 | 5.7 | 3.5 | 86.8 | |
| | 10 | 3.4 | 24.9 | 29.4 | 43.7 | | 5.3 | 5.6 | 4.3 | 85.1 | |
| | 50 | 4.9 | 4.1 | 15.6 | 77.4 | G₁ abrogation | 14.3 | 10.2 | 11.3 | 64.9 | Cell Death |
| MK-2 | 0 | 1.6 | 16.4 | 31.1 | 52.1 | | 7.0 | 5.9 | 2.2 | 85.1 | |
| | 50 | 4.0 | 19.0 | 27.8 | 50.1 | | 5.9 | 6.1 | 2.8 | 85.5 | |
| | 100 | 3.5 | 18.4 | 23.0 | 55.8 | | 5.5 | 6.1 | 3.3 | 85.4 | |
| | 250 | 6.9 | 11.2 | 10.0 | 71.9 | | 8.1 | 5.4 | 2.8 | 83.9 | |
| | 500 | 5.4 | 3.4 | 2.9 | 88.4 | G₁ abrogation | 11.8 | 4.4 | 4.1 | 80.0 | Cell Death |
| MK-4 | 0 | 1.9 | 20.2 | 29.7 | 50.0 | | 8.7 | 5.7 | 2.0 | 83.9 | |
| | 50 | 1.8 | 21.2 | 28.5 | 50.3 | | 8.9 | 6.2 | 2.8 | 82.3 | |
| | 100 | 2.4 | 22.0 | 27.4 | 49.7 | | 9.8 | 6.2 | 3.4 | 80.8 | |
| | 250 | 3.1 | 21.2 | 24.6 | 52.7 | | 9.3 | 5.8 | 3.1 | 82.2 | |
| | 500 | 5.0 | 18.2 | 16.0 | 61.8 | G₁ abrogation | 11.6 | 5.2 | 5.4 | 78.2 | Cell Death |

| | | HCT116 cells | | | | | HT29 cells | | | | |
|---|---|---|---|---|---|---|---|---|---|---|---|
| Agent | μM | sub G | G₁ | S | G₂ + M | | sub G | G₁ | S | G₂ + M | |
| Canthar- | 0 | 4.9 | 26.8 | 8.7 | 60.1 | | 5.9 | 40.7 | 9.2 | 44.7 | |
| idin | 1 | 4.2 | 26.1 | 13.8 | 58.2 | | 16.6 | 35.2 | 10.6 | 38.2 | |
| | 5 | 4.0 | 23.6 | 10.4 | 63.3 | | 5.3 | 38.6 | 10.6 | 46.3 | |
| | 10 | 4.2 | 25.7 | 9.4 | 62.2 | | 6.4 | 21.1 | 12.3 | 60.8 | |
| | 50 | 12.0 | 12.2 | 15.6 | 63.3 | G₁ abrogation | 14.7 | 23.0 | 20.7 | 43.1 | G₁ abrogation |
| MK-2 | 5 | 3.7 | 30.8 | 7.9 | 57.2 | | 17.3 | 31.8 | 8.7 | 41.3 | |
| | 50 | 3.3 | 32.8 | 6.3 | 57.3 | | 10.3 | 35.4 | 8.8 | 44.7 | |
| | 100 | 3.1 | 29.9 | 7.2 | 59.6 | | 3.5 | 40.6 | 9.0 | 45.9 | |
| | 250 | 6.2 | 23.9 | 4.3 | 65.4 | | 2.7 | 24.9 | 12.3 | 59.4 | |
| | 500 | 6.4 | 15.4 | 4.9 | 73.0 | G₁ abrogation | 8.8 | 24.1 | 20.4 | 45.2 | G₁ abrogation |
| MK-4 | 0 | 10.3 | 31.4 | 6.2 | 52.1 | | 7.0 | 35.1 | 9.3 | 48.4 | |
| | 50 | 6.3 | 26.7 | 5.8 | 61.3 | | 6.8 | 28.9 | 16.4 | 48.4 | |
| | 100 | 3.3 | 18.4 | 9.7 | 69.3 | | 6.3 | 33.3 | 17.2 | 43.3 | |
| | 250 | 8.2 | 16.3 | 8.2 | 67.8 | | 10.3 | 35.2 | 17.3 | 36.5 | |
| | 500 | 14.9 | 13.1 | 10.6 | 61.9 | G₁ abrogation | 3.9 | 39.4 | 19.2 | 37.7 | G2 abrogation |

(c) Combination Studies:

The cell lines listed above are exposed continuously to cisplatin and the phosphatase inhibitor in various drug ratio combinations for 72 h and then assayed for cytotoxicity; Similarly, the cells are exposed to 8 Gy of radiation and incubated with the phosphatase inhibitor and assessed for cytotoxicity at 72 h.

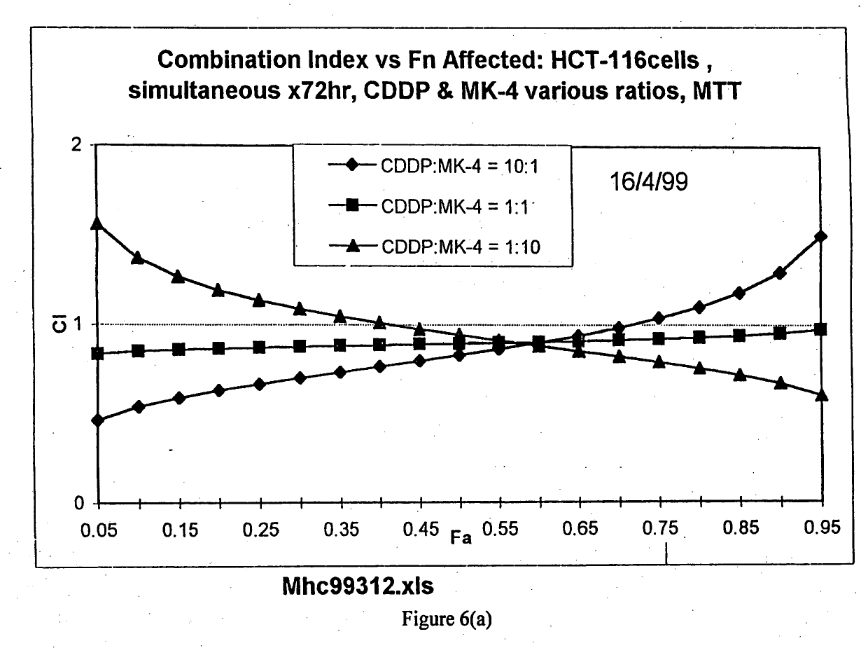

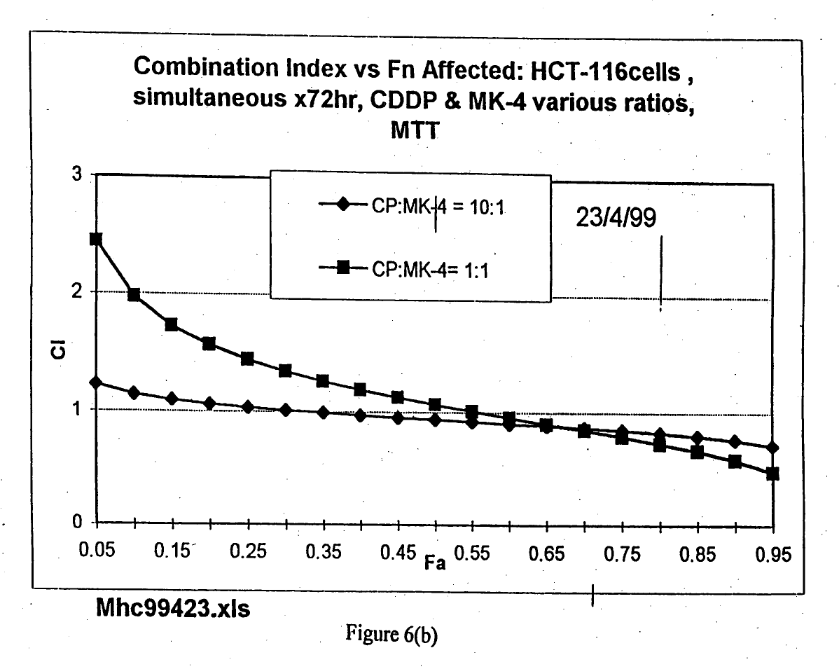

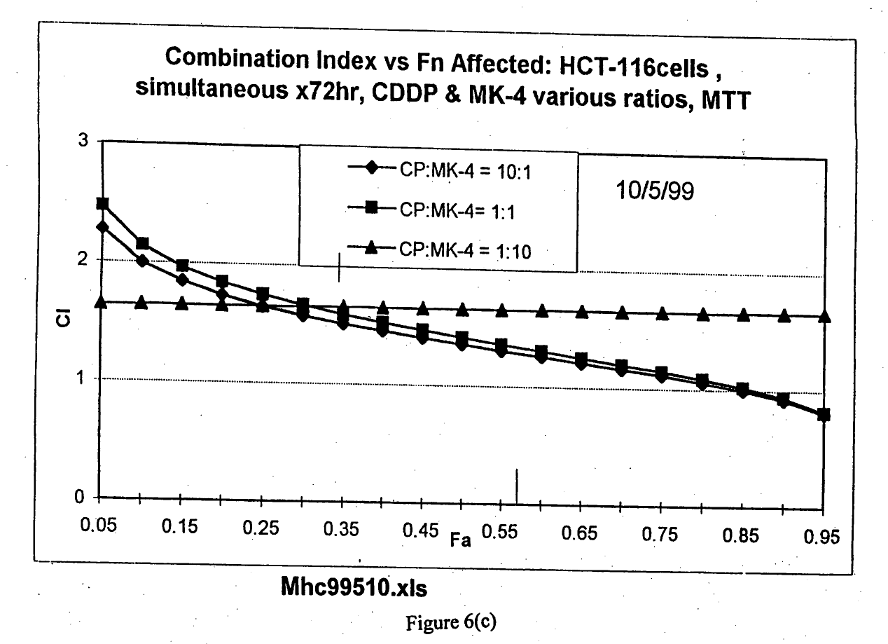

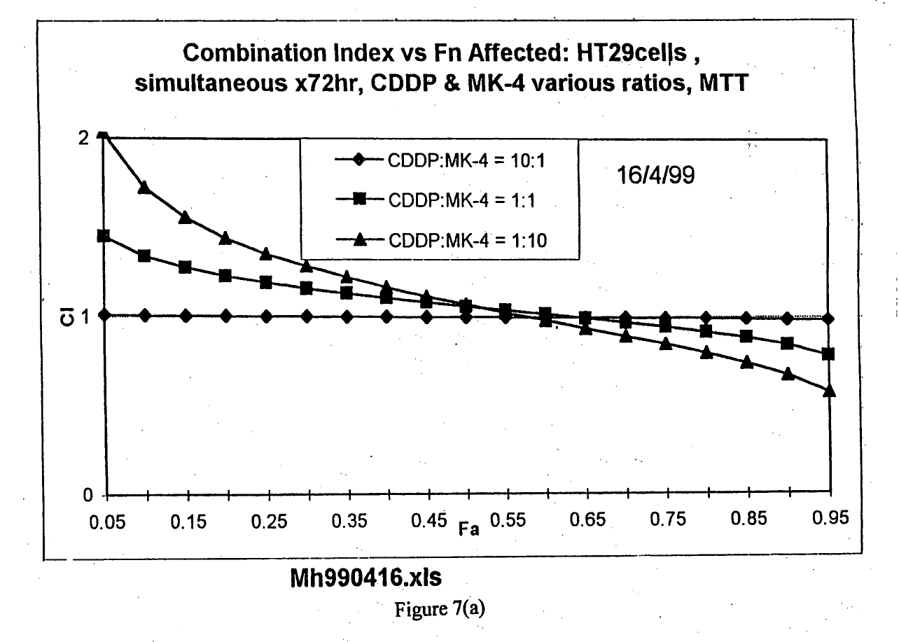

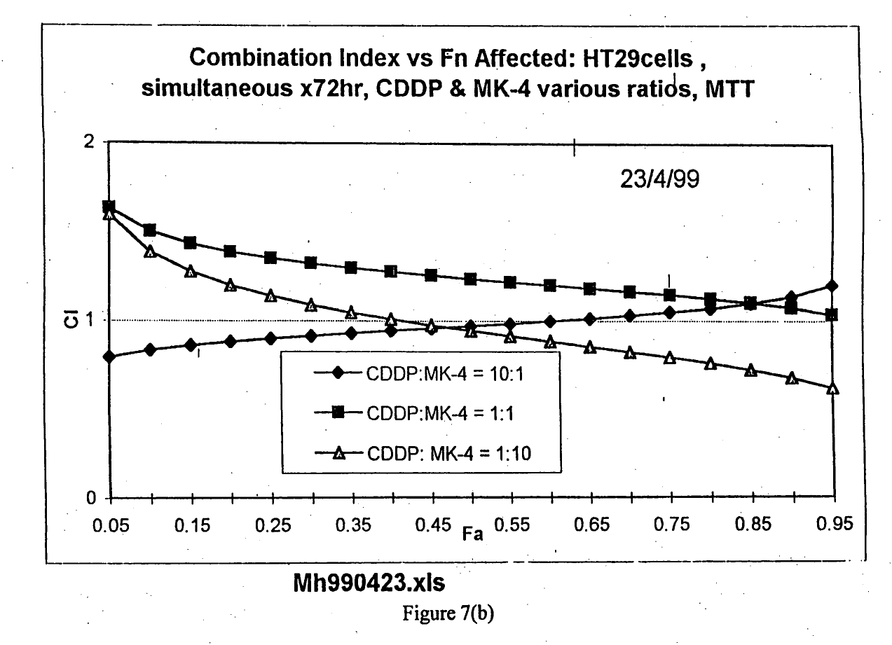

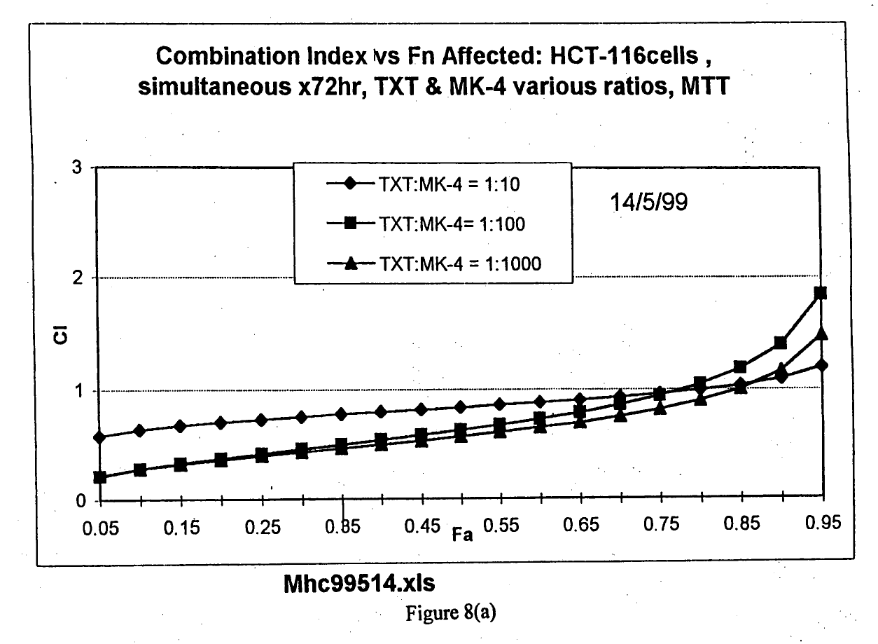

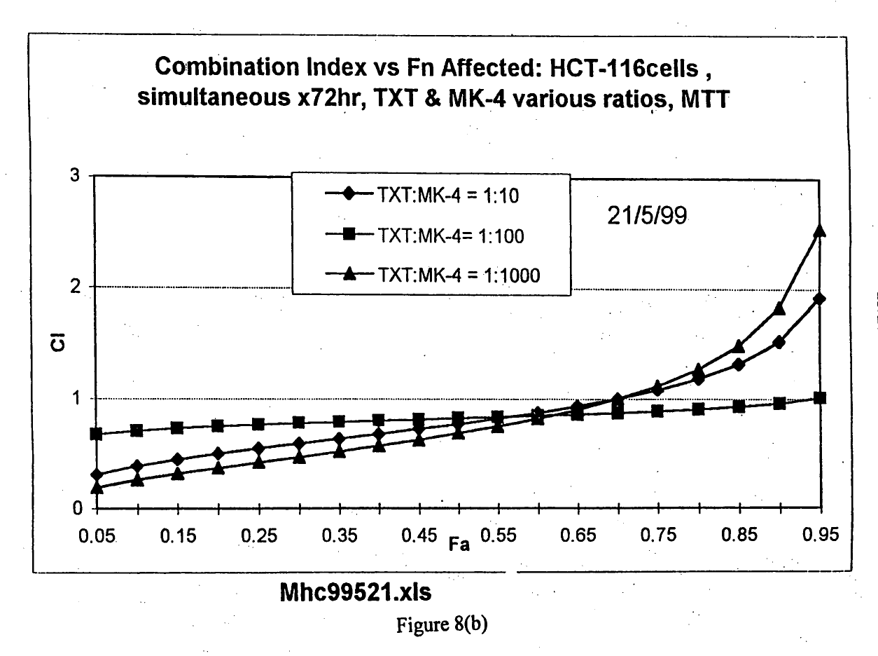

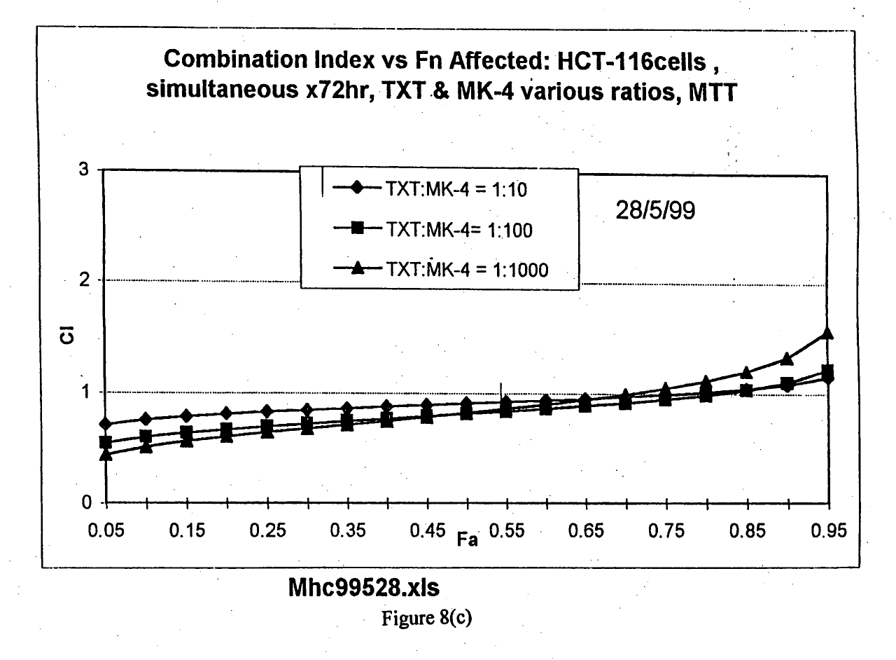

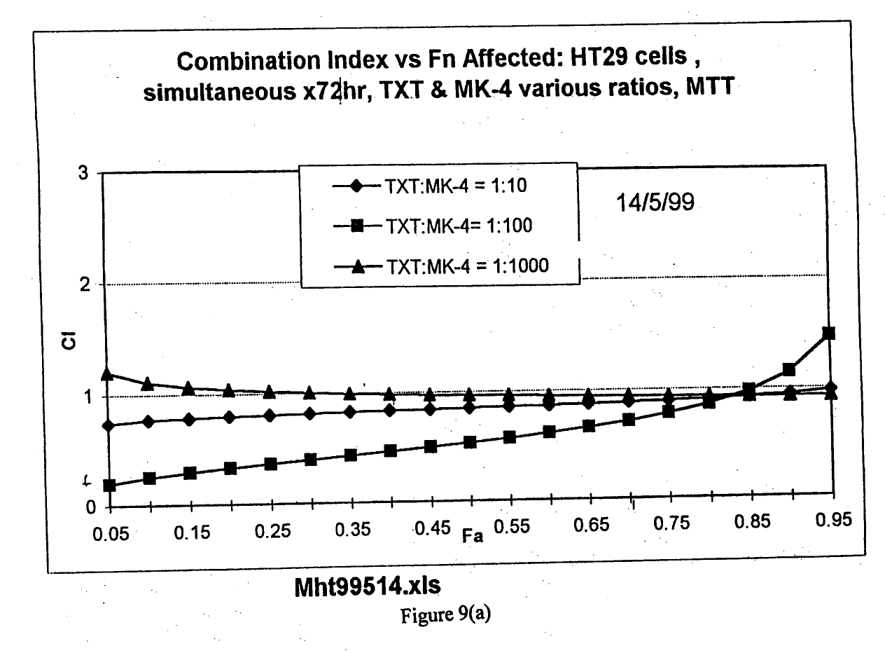

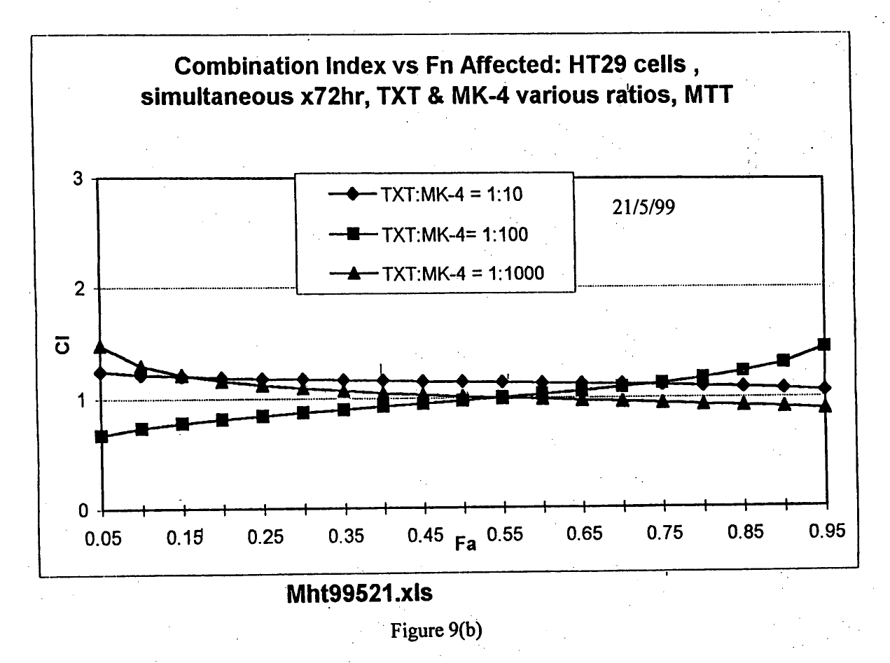

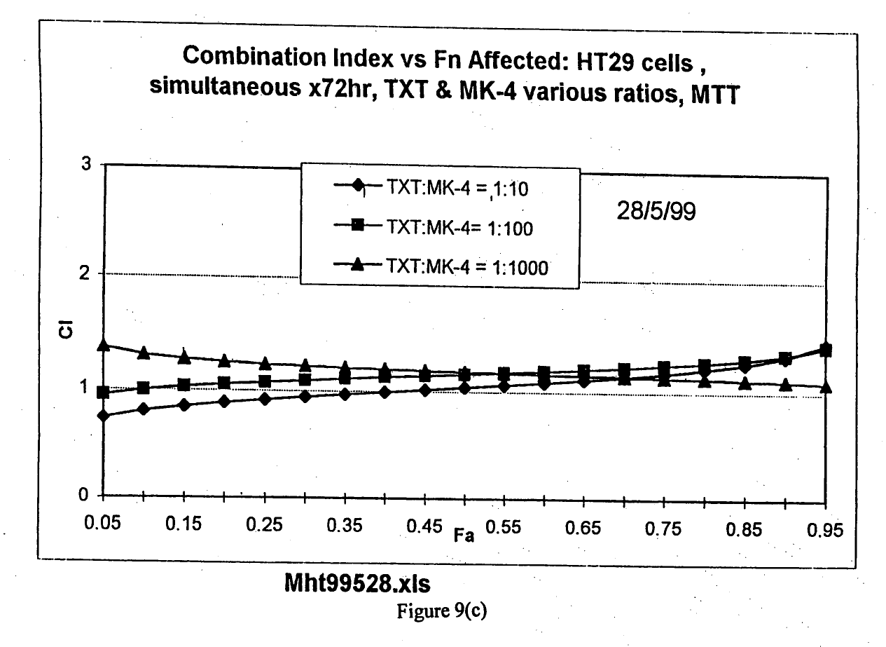

Data shown in FIGS. 6-9 shows the results of combination studies utilising the Median Effect Method in HT29 and HCT116 human colon cells. This method tests the cytotoxicity of various drug combinations from which a combination index can be calculated. A value of greater than one indicated antagonism, a value equal to 1 indicates additivity, while a value less than one indicates synergism. The HT29 and HCT116 cell lines were chosen as they have differing p53 status and they represent the tumour types that responded the greatest to cantharidin and its analogues.

The data show that the simultaneous combination of cisplatin and MK-4 in both HCT116 and HT29 cells was additive and not synergistic using drug molar ratios of 1:1, 10:1 and 1:10. An additive response indicated that the drugs were mediating their effects via two separate biochemical pathways. The simultaneous combination of taxotere and MK-4 in HT29 cells was also additive using drug molar ratios of 1:10, 1:100, 1:1000 (Taxotere: MK-4). However, this drug combination of taxotere and MK-4 induced a synergistic response in HCT116 cells. A synergistic response indicates that the two drugs were interacting in such a way as to enhance the overall cytotoxic response and to induce “more than the additive” response of each individual agent. Consequently, the addition of subtoxic levels of MK-4 clearly enhanced the cytotoxicity of taxotere.

### EXAMPLE 8

Results and Discussion

Anhydrides and simple analogues were synthesised according to literature procedures (Eggelte et. al; 1973), and then subjected to a PP1 and PP2A bio-assay (see biochemistry) to determine their ability to inhibit these enzymes. The results of initial screening at 100 mMs are shown in Table 4, along with IC₅₀ values in some instances.

TABLE 4
The inhibition of protein phosphatase 1 and 2A by anhydride modified
cantharidin analogues.

| | | | Selec- |
|---|---|---|---|
| | | | tivity |
| | Inhibition of | Inhibition of | PP2A/ |
| Compound | PP1 (%) | PP2A (%) | PP1 |
| 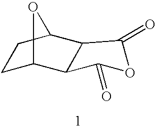 | 90 IC₅₀ 2.4 μM | 97 IC₅₀ 2.1 μM | 0.875 |
| 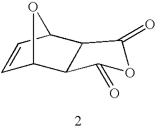 | ND | 95 | |
| 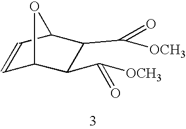 | 46 IC₅₀ 50 μM | 6 IC₅₀ >10,000 μM | >200 |
| 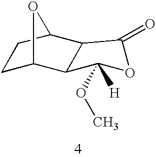 | 13 | 11 | |
| 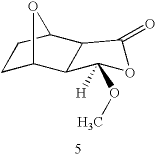 | 15 | 8 | |
| 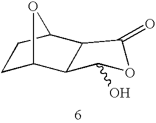 | 9 | 11 | |
| 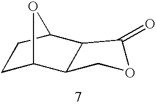 | ND | 21 | |
| 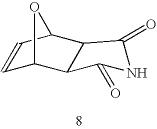 | ND | 15 | |
| 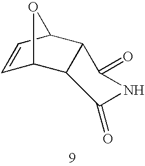 | ND | 4 | |

Of the compounds listed in Table 4, only 1 and 2 show any significant inhibition of PP2A. at 97% and 95% respectively (with little selectivity apparent for either enzyme). Interestingly the bioisoseteric replacement of the anhydride oxygen atom of 1 results in a complete loss of inhibition. Indeed no modification of the cyclic anhydride, is tolerated, and consequently results in no inhibition of PP2A.

Previously we have shown that analog 2 undergoes a rapid conversion to the dicarboxylic acid under assay conditions. We thus examined the stability of the non-active analogues (in Table 4) and found that they were stable under assay conditions showing no decomposition, in fact 5 can be synthesised via the Diels-Alder reaction in water (Eggelte et al; 1973).

In all instances, the corresponding dicarboxylic acid derivatives display lower inhibitory values at PP2A (Tables 5 and 6). Even though the anhydrides undergo a facile ring opening to the dicarboxylic acids, the original conformation presented at the active site must also play a role in determining the overall level of inhibition. Consequently, we believe that the conformation of anhydride carbonyl groups is more favourable for inhibition (essentially only one conformation presented at the active site), than that of the dicarboxylic acid (four possible minimum energy conformations, data not shown).

TABLE 5
Effects of anhydride to dicarboxylic acid on the inhibition of PP2A

| | | Inhibition | | Inhibition |
|---|---|---|---|---|
| Entry | Anhydride | (%) | Carboxylic acid | (%) |
| 1 | 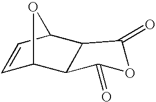 | 97 (This work) | 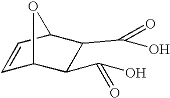 | 80 |
| 2 | 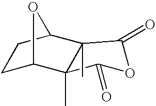 | 92-95 | 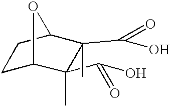 | 92-95 |
| 3 | 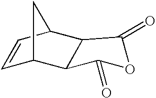 | 48 | 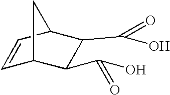 | 17 |

In an attempt to determine the feasibility of anhydride opening via nucleophilic attack from Tyr272, we conducted a series of model experiments in which 2 was allowed to stand in a chloroform solution of phenol. This mixture was examined periodically by ¹H NMR spectroscopy and showed the growth of a new species over a period of time (ca 10 days). Further analysis indicated the presence of a phenolate ester of norcantharidin (scheme 4). Consequently, a metal assisted or nucleophilic attack under physiological conditions represents a possible mode of assisted ring opening with the anhydride held in a favourable conformation within the active site. In turn the resultant diacid rapidly binds in a more favourable manner.

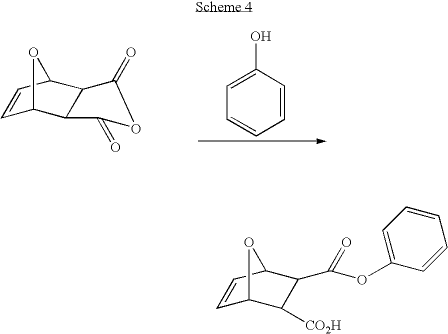

TABLE 6
Inhibition of PP1 and PP2A by selected cantharidin analogues

| | | Inhibition of | Inhibition of | Selectivity |
|---|---|---|---|---|
| Entry | Compound | PP1 (%) | PP2A (%) | PP2A/PP1 |
| 1 | 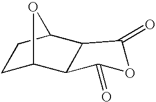 | 90 (IC₅₀ 2.4 μM) | 97 (IC₅₀ 2.1 μM) | 0.875 |
| 2 | 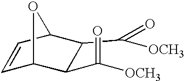 | 46 (IC₅₀50 μM) | 6 (IC₅₀ >10000 μM) | >200 |
| 4 | 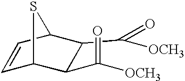 | 3 | 3 | Not determined |
| 5 | 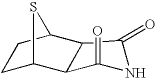 | 15 | 69 | Not determined |

The results presented herein indicate that cantharidin analogues, via anhydride opening are more potent inhibitors of PP2A. Analogues in which the anhydride moiety has been modified preventing a facile ring opening (except where otherwise indicated) are extremely poor inhibitors of PP2A (Tables 5 and 6).

However, the most interesting result reported herein (see table 4) is the selective inhibition of PP1 by the dimethyl ester (3). Simple diesterification of 2 has completely reversed the previously reported PP2A selectivity (ca 10 fold) of norcantharidin for PP2A to yield selective small synthetic molecule for the inhibition of either PP1 or PP2A. Again this suggests that presentation of a diacid moiety to the active site is crucial for the inhibition of PP2A. No such restrictions are apparent with the limited structure activity data for PP1.

A synthetic inhibitor such as 3 represents a significant advance on the currently widespread inhibitors of PP1 and PP2A.

In conclusion, the present inventors have demonstrated that a facile ring opening of the anhydride moiety is relevant for inhibition at PP2A. Also, that modification of the dicarboxylic acid moiety gives rise to a PP1 selective compound.

The above describes some embodiments of the present invention. Modifications obvious to those skilled in the art can be made without departing from the scope of this invention.

### INDUSTRIAL APPLICABILITY

It should be clear that the present invention will find light applicability, especially in the medical and veterinary fields.

### REFERENCES

Walsh A H, Chen A T, Honaken R E, FEBS Lett., 1997, 416, 230
Roberge M, Tudan C, Hung S M F, Harder K W, Jirik F R, and Anderson H Cancer Research 1994, 54, 6115-6121.
McCluskey, A; Taylor, C; Quinn. R J; Suganuma, M; Fujiki, H; Bioorg. Med. Chem. Lett., 1996, 6, 1025.
McCluskey A, Keane M A, Sim A T R, J. Med. Chem. 1998, submitted.
Enz, A; Zenke, G; Pombo-Villar; Bioorg. Med. Chem. Lett., 1997, 7, 2513; and
Sodeoka, M; Baba, Y; Kobayashi, S; Hirukawa, N; Bioorg. Med. Chem. Lett., 1997, 7, 1833.
Cohen, P; Cohen, PTW; J. Biol. Chem., 1989, 264, 21435.
Cohen, P; “The Structure and Regulation of Protein Phosphatases” Raven Press: New York, 1990, p230-235.
Shenolinkar, S; Maim, A C; Adv. Second Messenger Phosphoprotein Res., 1991b, 23, 1.
Krebs, E G; Angew. Chem. Int. Ed. Engl., 1993, 32, 1122.
Fischer, E; Angew. Chem., Int. Ed. Engl., 1992, 32, 1130.
Tachibana, K; Scheuer, P J; Tsukitani, Y; Kikuchi, H; Van Engen, D; Clardy, J; Gopichand, Y; Schmitz, F J; J. Am. Chem. Soc., 1981, 103, 2469.
Cheng, X-C; Kihara, T; Kusakabe, H; Magae, J; Kobayashi, Y; Fang, R-P; Ni, Z-F; Shen, Y-C; Ko, K; Yamaguchi, I; Isono, K; J. Antibiol., 0.1987, 40, 907.
Botes, D P; Tuinman, A A; Wessels, P L; Viljoen, C C; Kruger, H; Williams, D H; Santikarn, S; Smith, K J; Hammond, S J; J. Chem. Soc. Perkin Trans 1, 1984, 2311.
Herada, K-I; Matsuura, K; Suzuki, M; Oka, H; Watanabe, M F; Oishi, S; Dahlem, A M; Beasley, V R; Carmichael, W W; J. Chromatog., 1998, 448, 275.
Honaken, R E; FEBS Lett, 1993, 330, 283.
Matszawa, S; Suzuki, T; Suzuki, M; Matsuda, A; FEBS Lett., 1994, 356, 272.
Wang, G-S; J. Ethnoparmacol., 1989, 26, 147.
Cavill, G W K; Clark, D V; Naturally Occuring Insecticides (M. Jacobson and D G Crosby, EDS), Marcel Dekker, New York, 1971, 271.
Oaks, W W; DiTunno, J F; Magnani, T; Levey, H A; Mills, L C; A.M.A. Arch. Int. Med., 1960, 105, 106.
Goldfarb, M T; Gupta, A K; Sawchuk, W S; Dermatologic Clinics, 1991, 9, 287.
A. Schmitz, D G; J. Vet. Intern. Med., 1989, 3, 208.
Polettini, A; Crippa, U; Ravagii, A; Saragoni, A.; Forensic. Sci. Int., 1992, 56(1), 37.
Li, Y-M; Casida, J E; Proc. Natl. Acad. Sci. USA, 1992, 89, 11867.
McCluskey, A; Taylor, C; Quinn, R J; Suganuma, M; Fujiki, H; Bioorg. Med. Chem Lett., 1996, 6, 1025.
Eggelte, T A; de Koning, H; Huisman, H O; Tetrahedron, 1973, 29, 2445; 3 see: Tamura, Y; Imanishi, K; Miki, Y; Ikeda, M; Synthesis, 1997, 559; 5 & 9 see: Kwart, H; Burchuk, I; J. Am. Chem. Soc., 1952, 74, 3094.
Goldberg, J; Huang, H-B; Kwon, Y-G; Greengard, P; Nairn, A C; Kuriayan, J; Nature, 1995, 376, 745.
Quinn, R J; Volter, K; Embury, K J, unpublished results.
Cohen, P; Alemany, S.; Hemmings, B A; Resink, T J; Stralfors, P and Tung, H Y L in Methods in Enzymology (Ed. J D Corbin and R A Johnson) 1988, 159, 390. Academic Press (London).
Hardie, D G, Haystead T A J; Sim A T R; Protein Phosphorylation, in methods in enymology, in press. Enz. A; Zenke, G; Pombo-Villar; Bioorg. Med. Chem. Lett., 1997, 7, 2513.
Sodeoka, M; Baba, Y; Kobayashi, S; Hirukawa, N; Bioorg. Med. Chem. Lett., 1997, 7, 1833.
Durfee T., et al., (1993). The retinoblastoma protein associates with the protein phosphatase type 1 catalytic subunit. Genes Dev. 7, 555-569.
Ghosh, S. et al. (1996). Okadaic acid overrides the S-phase checkpoint and accelerates progression of G2-phase to induce premature mitosis in Hela cells. Exp. Cell Res. 227:165-169.
Herwig, S., and Strauss. M. (1997). The retinoblastoma protein: a master regulator of cell cycle, differentiation and apoptosis. Eur.J.Biochem. 246:581-601.
Jackman, A L and Calven. AH (1995). Folate-based thymidylate synthase inhibitors as anticancer drugs. Ann. Oncol. 6:871-881, 1995.
Kawamura K-I, Grabowski D, Weizer K, Bukowski R, and Ganaphthi R (1996). Modulation of vinblastine cytotoxicity by dilantin (phentoin) or the protein phosphatase inhibitor okadaic acid involves the potentiation of anti-mitotic effects and induction of apoptosis in human tumour cells. Br. J. Cancer 73: 183-188.
Lazzereschi, D. et al. (1997). The phosphatase inhibitor okadaic acid stimualtes the TSH-induced G1-S phase transition in thyroid cells. Exp. Cell Res. 234:425-433. Nakamura K. and Antoku S. (1994). Enhancement of X-ray cell killing in cultured mammalian cells by the protein phosphatase inhibitor calyculin A. Cancer Res 54:2088-20990.
O'Connor P M (1996). Cell cycle checkpoints: Targets for anti-cancer therapy. Anti-Cancer Drugs 7(sup 3): 135-41.
O'Connor P M (1997). Mammalian G1 and G2 phase chekpoints. Cancer Surveys 29:151-182.
Peters G J and Ackland S P (1996). New antimetabolites in preclinical and clinical development. Exp. Opin. Invest. Drugs 5, 637-679.
Sch\check{s}nthal A (1992). Okadaic acid—a valuable new tool for the study of signal transduction and cell cycle regulation? New Biol. 4, 16.
Stein, G. S. et al. (1998). The molecular basis of cell cycle and growth control. Wiley-Liss Publishers. New York.
Yamashita K. et al (1990). Okadaic acid, a potent inhbitor of type 1 and type 2A protein phosphatases, activates cdc2/H1 kinase and transiently induces a premature mitosis-like state in BHK21 cells. EMBO J. 9: 43314338.

1. A cell permeable inhibitor of protein phosphatase, said inhibitor being an anhydride modified cantharidin analogue.

2-3. (canceled)

4. A compound of the formula: 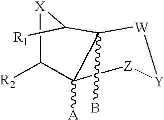
wherein R₁ and R₂ are H, aryl or alkyl; X is O, N or S; Y is O, S, SR, NH, NR, CH₂OH, CH₂OR; R is alkyl or aryl; A and B are H or CH₃; W and Z are CHOH or C=0 and R₁ and R₂ can cyclise to form a ring as follows: 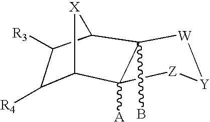
wherein R₃ and R₄ are H, aryl or alkyl.

5-12. (canceled)

13. A method of treating cancer which method comprises administering to a patient in need of such treatment, an effective amount of an inhibitor according to any one of claims 1 to 3 or a compound according to any one of claims 4 to 6, together with a pharmaceutically acceptable carrier, diluent and/or excipient.

14. A method according to claim 13, wherein the cancer is inherently resistant to conventional chemotherapy.

15. A method according to claim 13 or claim 14, wherein the cancer is colon cancer or non small-cell lung cancer.

16. A method according to any one of claims 13 to 15, wherein the inhibitor or the compound is administered intravenously.

17-29. (canceled)
